(19) United States
(12) Patent Application Publication
(10) Pub. No.: US 2026/0131081 A1
(43) Pub. Date: May 14, 2026

(54) PRE-FILLED SYRINGE WITH EMBEDDED FILTER

(71) Applicant: Genentech, Inc., South San Fransisco, CA (US)

(72) Inventors: Kunjal Oza, Danville, CA (US); Renato Ravanello, South San Francisco, CA (US); Christopher Arena, San Francisco, CA (US); Rachel Paige Arnott, San Francisco, CA (US); Patrick James Grandt, Berkeley, CA (US); Kristof William Roham Hovaten, San Leandro, CA (US); David Ellis Overcashier, Half Moon Bay, CA (US)

(73) Assignee: Genentech, Inc., South San Fransisco, CA (US)

(21) Appl. No.: 19/293,142

(22) Filed: Aug. 7, 2025

Related U.S. Application Data
(63) Continuation of application No. PCT/US2024/015924, filed on Feb. 15, 2024.
(60) Provisional application No. 63/445,922, filed on Feb. 15, 2023.

(51) Int. Cl. A61M 5/31; A61M 5/315; A61M 39/00; A61M 39/24

(52) U.S. Cl. CPC A61M 5/3145; A61M 5/31578; A61M 5/3159; A61M 39/24; A61M 2039/0009; A61M 2039/2426; A61M 2202/0007; A61M 2202/04; A61M 2205/3331; A61M 2205/7545; A61M 2207/00

(57) ABSTRACT

Apparatus and methods for filtering syringe fluid contents through a syringe-embedded filter. The filter may be sealed within a housing lodged distal to unfiltered fluid contents, proximal a syringe distal outlet, with a housing exterior sealed fluid-tight against a syringe inner wall. Before filtering, the housing may block access of unfiltered fluid to the filter. The unfiltered fluid may be sealed between a syringe plunger, the inner wall and the housing. Distally directed urging of the unfiltered fluid may unblock the housing to a flow of unfiltered fluid to the filter. Fluid-tight sealing within the housing may limit fluid passage to the distal outlet to a path through the filter. The unfiltered fluid may be urged through the unblocked housing and the filter. Filtered fluid, relatively free of particulate matter sized greater than a filter average pore size, may flow to the distal outlet for delivery to a target site.

### BACKGROUND

A pre-filled syringe (PFS) is often used to deliver medicament to a target site. The PFS offers a user several advantages over a conventional syringe. Beyond elimination of a user-step of filling the syringe with the medicament, these advantages usually also include improved accuracy and consistency of dosing; decreased pre-delivery exposure of a delivery needle to a non-sterile environment; and reduced potential for inadvertent needle sticks.

However, some disadvantages may attend use of the PFS. PFSs are typically intended for use after a period—possibly an extended period—of storage. During storage, contact adhesions may form between a syringe plunger of the PFS and an inner wall of a syringe barrel of the PFS. The adhesions may necessitate a high break-loose force being required to overcome such “stiction” to initiate movement of the plunger within the barrel. Over the storage period, molecular deposits may form along the inner wall. The adhesions and deposits may necessitate a high gliding force being required to maintain movement of the plunger within the barrel during medicament delivery. The high break-loose force and/or the high gliding force may be difficult for the user to achieve, sustain or regulate. Application by the user of such high forces may cause discomfort to a patient receiving a PFS injection.

To minimize complications involving adhesions/deposits, PFS manufacturing typically includes lubrication of the inner barrel wall and/or of the plunger's exterior. While generally improving requirements of break-loose force and gliding force, lubrication often entails other complications. Over time, materials used for the lubrication, typically silicone-oil based, can migrate from the lubricated surface(s) to form free-floating particles within liquid contents of the PFS. Such particles may further grow through self-aggregation and/or through accretion of medicament molecules.

Independent of lubricant-related complications, extended exposure of medicament to surfaces within the PFS and to molecules of the medicament, may eventuate particle-forming self-aggregation of the medicament molecules.

Most of the aforementioned particles, are deemed non-therapeutic and may possibly be detrimental to the patient.

The particles may be removed just prior to delivery by passing PFS contents through an appropriately pore-sized filter. A standard approach to filtration of syringe contents may include affixing the filter between the delivery needle and a distal delivery outlet of the syringe barrel just before medicament delivery to the target site. However, implementation of such an approach would likely work against many of the PFS advantages of safety and simplicity of use.

It would be desirable, therefore, to provide apparatus and methods for filtering PFS contents close in time before medicament delivery in a fashion that maintains the advantages of PFS use.

Manufacture of a PFS with an integrally attached filter may result, during the storage period, in fouling of the filter with medicament and/or with particles, accretions and aggregates. Such fouling may diminish ease of use and functionality of the filter during medicament delivery.

It would also, therefore, be desirable to provide apparatus and methods for filtering PFS contents close in time before medicament delivery in a fashion that sequesters PFS contents from the filter during PFS storage before the delivery; and that allows PFS contents access to the filter surface during the delivery.

### BRIEF DESCRIPTION OF THE DISCLOSURE

Apparatus and methods are provided for filtering PFS contents close in time before the medicament delivery in a fashion that maintains the advantages of PFS use.

Apparatus and methods are provided for filtering PFS contents close in time before the medicament delivery in a fashion that sequesters medicament from the filter surface during PFS storage before the delivery; and that allows access to the surface by readily flowing medicament to the filter during the delivery.

Apparatus and methods directed to the above are provided as disclosure of a filter embedded within a PFS near a distal delivery outlet of the PFS, with the filter closed off from proximally stored medicament before the medicament delivery, and open to flow of the medicament during the medicament delivery.

Apparatus and methods directed to the above are provided as disclosure of a filter housing lodged within a PFS near the distal delivery outlet of the PFS, with the filter housing—prior to the medicament delivery—preventing medicament access to the filter housed in the filter housing; and with the filter housing—during the medicament delivery—allowing the medicament access to the filter.

The methods may include methods of manufacturing the filter housing. The methods may include methods of manufacturing the pre-filled syringe.

### BRIEF DESCRIPTION OF THE DRAWINGS

The objects and advantages of the invention will be apparent upon consideration of the following detailed description, taken in conjunction with the accompanying drawings, in which like reference characters refer to like parts throughout, and in which:

FIG. 1 is a schematic view of apparatus in accordance with principles of the invention;

FIG. 2 is an isometric view of apparatus in accordance with principles of the invention, with select exterior features shown in partial cross-section to provide an unobstructed view of interior features;

FIG. 3 is an exploded isometric view of apparatus in accordance with principles of the invention;

FIG. 4 is an isometric view of apparatus in accordance with principles of the invention, with select features shown in partial cross-section;

FIG. 5 is a partial cross-sectional view of apparatus in accordance with principles of the invention;

FIG. 6 is a sectional view of the apparatus shown in FIG. 5, the view taken along line 6-6 (shown in FIG. 5);

FIG. 7 is an isometric view of apparatus in accordance with principles of the invention, with select features shown in partial cross-section, the view showing a stage of use subsequent to the view shown in FIG. 6;

FIG. 8 is a partial cross-sectional view of the apparatus shown in FIG. 7, the view taken along line 8-8 (shown in FIG. 7);

FIG. 9 is an isometric view of apparatus in accordance with principles of the invention, with select exterior features shown in partial cross-section to provide an unobstructed view of interior features;

FIG. 10 is an exploded isometric view of apparatus in accordance with principles of the invention;

FIG. 11 is a partial cross-sectional view of apparatus in accordance with principles of the invention;

FIG. 12 is a partial cross-sectional view of the apparatus shown in FIG. 11, the view taken along line 12-12 (shown in FIG. 11);

FIG. 13 is a partial cross-sectional view of apparatus in accordance with principles of the invention, the view taken from the same vantage as FIG. 12 and showing a stage of use subsequent to the view shown in FIG. 12; and

FIG. 14 is an isometric view of apparatus in accordance with principles of the invention, with select features shown in partial cross-section, the view showing a stage of use subsequent to the view shown in FIG. 13.

### DETAILED DESCRIPTION

Apparatus and methods for a filter embedded in a syringe are provided. The apparatus may include the syringe with the filter embedded in the syringe. The apparatus may include a filter housing with the filter affixed within the housing. The housing may be lodged in the syringe. The syringe may be a PFS.

The methods may include methods of manufacture of the apparatus.

The apparatus may include a fluid delivery device. The device may deliver a filtered fluid. The device may be configured for delivery of the filtered fluid. The filtered fluid may be delivered from a distal outlet of the device. The device may define a longitudinal axis.

The device may include a fluid container. The container may be disposed coaxial with the longitudinal axis. The container may contain a fluid. The fluid may include a medicament. The fluid may be filtered through the filter to deliver the filtered fluid.

The filter may have an average pore size. The average pore size may be from about 0.01 micron to about ten microns. The average pore size may be from about 0.01 micron to about one micron. The average pore size may be from about one micron to about ten microns. The filter may have any suitable average pore size. Any suitable average pore size may include an average pore size of 0.1 microns to about 0.3 micron. Any suitable average pore size may include an average pore size of about 4 microns to about 6 microns.

Any suitable average filter pore size may include an average pore size selected to be smaller than an average effective diameter of particulate matter to be filtered from the unfiltered fluid. The average effective diameter of particulate matter to be filtered from the unfiltered fluid may be about 0.4 micron. The average effective diameter of particulate matter to be filtered from the unfiltered fluid may be about 7 microns.

Also anticipated is use in the invention of a specialty filter configured to retain matter on a basis of the matter's charge. Also anticipated is use in the invention of a specialty filter configured to retain matter through binding of the matter to specific molecular species affixed onto and/or into the specialty filter. Such species may include polypeptides. The polypeptides may include portions of antibodies. Such species may include polynucleotide strands. The polynucleotide strands may include portions of DNA. The polynucleotide strands may include portions of RNA. The specialty filter may be used to remove, from PFS fluid contents, matter that is smaller (in at least a direction of fluid flow through the specialty filter) than a pore size of the specialty filter. The specialty filter may also retain matter on a basis of size.

The container may include a syringe barrel. The syringe barrel may define a barrel axis. The barrel axis may be disposed coaxial with the longitudinal axis.

The syringe barrel may include a wall. The wall may include a polymer. The polymer may include a plastic polymer. The wall may include glass. The glass may include a borosilicate glass.

The wall may include an inner wall. The inner wall may be disposed coaxially with the longitudinal axis. The inner wall may border the fluid. The inner wall may radially constrain the fluid.

The device may include the filter. The filter may be embedded within the container. The filter may be disposed proximal the distal outlet. The filter may be disposed athwart the longitudinal axis. The filter may be disposed between the fluid and the distal outlet.

The device may include the filter housing. The filter may be fixed in the filter housing. The filter housing may be disposed within the container. The filter housing may be disposed proximal the distal outlet. The filter housing may be disposed between the fluid and the distal outlet. The filter housing may be lodged in the container. The filter housing may be configured to maintain the filter oriented athwart the longitudinal axis.

The filter housing may include embedment elements that seal against the inner wall. The embedment elements may include external ribs. The external ribs may lie along an exterior of the filter housing. The exterior of the filter housing may include a contour that is complementary to a contour of the inner wall. The exterior of the filter housing may have a convex contour that is complementary to a concave contour of the inner wall. The embedment elements may be configured to seal against the inner wall. The embedment elements may be configured to prevent passage of the fluid between the filter housing and the inner wall.

The filter housing may include a proximal filter cap. The filter housing may include a distal filter base. The filter may be maintained in place between the filter base and the filter cap. The filter may be maintained in place athwart the longitudinal axis by engagement of the filter base and the filter cap. The filter may be affixed to the filter base. The filter may be affixed to the filter cap.

The filter base may be configured to provide distal support to the filter. The distal support may be provided to a central area of the filter. The distal support may be provided by a proximal central projection of the filter base. A proximal face of the central projection may be disposed closely distal to the filter. A distal side of the central area of the filter may be disposed closely proximal to the central projection. The distal side of the central area of the filter may contact the central projection. The filter may be affixed to the central projection. The filter may not be affixed to the central projection.

The filter base may include a proximal rim. The proximal rim may support a proximal surface of the filter base. The filter may be affixed to the proximal surface. A periphery of the filter may be affixed to the proximal surface.

A region of the filter circumferentially bordering a filter perimeter may be joined to the proximal surface of the proximal rim. The region may be disposed on a distal face of the filter. The region may be disposed medial to the filter perimeter.

The region may be joined to the proximal surface of the filter base proximal rim in a fluid-tight seal. The region may be joined to the proximal surface by a weld. The weld may be produced by a laser welding process. The weld may be produced by a chemical welding process. The seal may extend circumferentially along the filter perimeter. The seal may extend circumferentially along all the filter perimeter. The seal may be configured to prevent passage of the fluid between an interior of the filter base and any of the filter perimeter.

Additionally or alternatively, the filter base and the filter cap may cooperate within an interior of the filter housing to provide a fluid-tight seal of the filter perimeter. The filter cap may include an interior filter cap circumferential sealing rib. The filter cap circumferential sealing rib may include a plurality of filter cap circumferential sealing ribs. The one or more than one filter cap circumferential sealing rib may be configured to extend distally within the interior of the filter housing. The filter base may include an interior filter base circumferential sealing rib. The filter base circumferential sealing rib may include a plurality of filter base circumferential sealing ribs. The one or more than one filter base circumferential sealing rib may be configured to extend proximally within the interior of the filter housing. A region of the filter medial to the filter perimeter may be sealed by a compression of the one or more than one filter cap circumferential sealing rib against the filter. The region of the filter may be sealed by a compression of the one or more than one filter base circumferential sealing rib against the filter. The region of the filter may be sealed by a compression of the filter between the one or more than one filter cap circumferential sealing rib and the one or more than one filter base circumferential sealing rib.

The filter base may be secured to the filter cap. An interference between a filter cap convexity and a filter base concavity may be configured to secure the filter cap to the filter base.

Facing surfaces of the filter cap convexity and the filter base concavity may be contoured complementarily to each other. The facing surfaces may feature complementary contouring. The complementary contouring may be approximate. The interference between the filter cap convexity and the filter base concavity may provide a fluid-tight seal. The fluid-tight seal may prevent passage of the fluid between the facing surfaces.

An interference between a filter cap concavity and a filter base convexity may be configured to secure the filter cap to the filter base. Facing surfaces of the filter cap concavity and the filter base convexity may be contoured complementarily to each other. The facing surfaces may feature complementary contouring. The complementary contouring may be approximate. The interference between the filter cap concavity and the filter base convexity may provide a fluid-tight seal. The fluid-tight seal may prevent passage of the fluid between the facing surfaces.

The filter cap convexity may be disposed along a portion of an interior surface of the filter cap. The filter cap concavity may be disposed along a portion of the interior surface of the filter cap. The interior surface of the filter cap may be a circumferential interior surface the filter cap.

The filter base concavity may be disposed along a portion of an exterior surface of the filter base. The filter base convexity may be disposed along a portion of the exterior surface of the filter base. The exterior surface of the filter base may be a circumferential exterior surface the filter base.

The filter cap convexity may be disposed along a portion of the exterior surface of the filter cap. The filter cap concavity may be disposed along a portion of the exterior surface of the filter cap. The filter base concavity may be disposed along a portion of an interior surface of the filter base. The filter base convexity may be disposed along a portion of the interior surface of the filter base.

The fluid-tight seal provided by the interference of the facing surfaces of the filter cap and of the filter base may be a circumferential seal. The circumferential seal may prevent passage of the fluid between the facing surfaces along all of a circumference of the filter housing. The circumferential seal may prevent passage of the fluid between the interior of the filter housing and an exterior of the filter housing.

A wall of the filter housing may be configured to seal against the inner wall of the container. The wall of the filter housing may be an exterior circumferential wall. The wall of the filter housing sealing against the container inner wall may prevent passage of the fluid between an exterior of the filter housing and the inner wall.

The wall may include a filter cap sealing flange. The filter cap sealing flange may be disposed along an external periphery of the filter cap. The filter cap sealing flange may include a plurality of filter cap sealing flanges.

The circumferential wall may include a filter base sealing flange. The filter base sealing flange may be disposed along an external periphery of the filter base. The filter base sealing flange may include a plurality of filter base sealing flanges.

The wall may include a filter base sealing surface. The sealing surface may be supported by a section of a body of the filter base. The section of the body of the filter base supporting the sealing surface may be a distal section of the body. The sealing surface may be disposed along a region of a conical distal exterior of the filter base. The sealing surface may be disposed along a region of a frustoconical distal exterior of the filter base.

The exterior of the filter base may feature a contour at least approximately complementary to a distal internal taper of the syringe barrel. The sealing surface may be disposed to lie against the distal internal taper. The distal internal taper may lie along a portion of the inner wall. The sealing surface may be configured to compress against the distal internal taper. The sealing surface may be configured to seal against the distal internal taper. The sealing surface sealing against the distal internal taper may prevent passage of the fluid between the distal exterior of the filter base and the distal internal taper. The sealing surface sealing against the distal internal taper may prevent passage of the fluid between the housing and the inner wall.

The container may enclose a volume of the fluid. The volume may be a pre-filled medicament volume of the PFS. The pre-filled medicament volume may include a medicament dosing volume. The pre-filled medicament volume may include a volume expected to be lost to pre-delivery syringe preparation. The volume expected to be lost to pre-delivery syringe preparation may include a volume expected to be lost to syringe priming. The volume expected to be lost to pre-delivery syringe preparation may include a volume expected to be lost to a filtering process. The filtering process may involve the filter. The filtering process may involve the filter housing.

The pre-filled medicament volume of the PFS may be sealed within the container by a syringe plunger disposed proximal to the fluid. The plunger may be configured to slidingly seal against the inner wall. The plunger may be configured to prevent proximal flow of the fluid between the inner wall and an exterior of the plunger.

The plunger may be configured to urge the fluid toward the distal outlet. Longitudinal displacement of the plunger toward the distal outlet may cause an urging of the fluid toward the distal outlet. To effect the medicament delivery, the user may displace the plunger longitudinally toward the distal outlet.

The device may include a plunger rod configured to move the plunger distally toward the distal outlet. To effect the medicament delivery, the user may displace the plunger rod longitudinally toward the distal outlet.

The plunger rod may be configured to move the plunger proximally away from the distal outlet.

Before the delivery of the filtered fluid, the pre-filled medicament volume may be disposed between the plunger and the filter. Before the delivery, the volume may be disposed between a distal face of the plunger and a proximal side of the filter.

Before the delivery of the filtered fluid, the pre-filled medicament volume may be disposed between the plunger and the filter housing. Before the delivery, the volume may be disposed between the distal face of the plunger and a proximal exterior side of the filter housing. Before the delivery, the volume may be bordered proximally by the distal face of the plunger. Before the delivery, the volume may be bordered distally by the proximal exterior side of the filter housing.

The filter cap may include the proximal exterior side of the filter housing. The filter cap may be configured, prior to the delivery, to block fluid access to the filter. The fluid may be configured, upon distally directed urging by the plunger, to unblock the filter cap to a flow of distally urged fluid.

Prior to the urging of the fluid toward the distal outlet, the filter housing may be configured to prevent the fluid from contacting the filter. The filter housing may be configured to prevent flow of the fluid to the filter prior to the urging. The filter housing may be configured to prevent access of the fluid to the filter prior to the urging.

Prior to the urging of the fluid toward the distal outlet, the proximal exterior side of the filter housing may be configured to prevent the fluid from contacting the filter. The proximal exterior side of the filter housing may be configured to prevent the flow of the fluid to the filter prior to the urging. The proximal exterior side of the filter housing may be configured to prevent access of the fluid to the filter prior to the urging.

An initiation of the urging of the fluid toward the distal outlet may increase fluid pressure upon the filter housing. An increase of the fluid pressure upon the filter housing may alter a configuration of the filter housing. The initiation of the distally directed urging may increase the fluid pressure upon the proximal exterior side of the filter housing. An increase of the fluid pressure upon the proximal exterior side of the filter housing may alter a configuration of the proximal exterior side of the filter housing. The increase of the fluid pressure upon the proximal exterior side of the filter housing may alter a configuration of a proximal side of the filter housing.

Following the initiation of the urging, the filter housing may be configured to allow access of the fluid to the filter. The filter housing may be configured to allow the flow of the fluid to the filter following the initiation of the urging. The filter housing may be configured to allow the fluid to contact the filter following the initiation of the urging.

Following the initiation of the urging, the proximal side of the filter housing may be configured to allow access of the fluid to the filter. The proximal side of the filter housing may be configured to allow the flow of the fluid to the filter following the initiation of the urging. The proximal side of the filter housing may be configured to allow the fluid to contact the filter following the initiation of the urging.

The filter cap may include a cylindrical body. The cylindrical body may be disposed coaxially with the longitudinal axis. Such coaxial disposition may be approximate. The cylindrical body may have an interior that includes an interior of the distal cap. The cylindrical body may have an internal wall. The internal wall may be a cylindrical wall.

In an embodiment of the filter cap, the cylindrical body may include a collar disposed at a proximal end of the body. Along an interior surface of the collar, the collar may include a collar rim. The rim may extend radially inward.

The proximal side of the filter housing may include a proximal plug of the filter cap. The proximal plug may include a circumferential plug groove disposed along an exterior of the plug. The circumferential groove may extend radially inward into a body of the plug. The groove may have a concave contour complementary to a radially inward-facing convex contour of the collar rim. The plug and the rim may be configured to compress against each other. The concave contour of the groove and the convex contour of the rim may be configured to compress against each other. Compression of the groove and the rim against each other may produce an interference between the plug and the collar. The interference between the plug and the collar may provide a force maintaining the plug in the collar.

The interference between the plug and the collar may provide a seal against flow of the fluid between the plug and the collar. Prior to the initiation of the urging of the fluid toward the distal outlet, the seal between the plug and the collar may prevent flow of the fluid into the interior of the cylindrical body. Prior to the initiation of the urging, the seal between the plug and the collar may prevent flow of the fluid into the interior of the filter cap. Prior to the initiation, the seal may prevent flow of the fluid into the interior of the filter housing.

The seal between the proximal plug and the collar may be configured to be overcome by the urging. The seal between the plug and the collar may be configured to be overcome by the initiation of the urging. The seal may be configured to be overcome by the increase, attendant upon initiation of the urging, of the fluid pressure upon the proximal exterior side of the filter housing.

The increase of the fluid pressure upon the proximal exterior side of the filter housing, may apply a distally directed force to the proximal plug. The distally directed force on the plug may be greater than the force maintaining the plug in the collar. The distally directed force on the plug may distally displace the plug, relative to the collar, into the interior of the filter cap.

With the increase of the fluid pressure upon the proximal exterior side of the filter housing, the proximal plug may undergo a distal displacement, relative to the collar, into the interior of the filter housing. The distal displacement of the plug into the interior of the filter housing may unseal the collar to the flow of the fluid through the unplugged collar, the flow continuing into the interior of the filter housing and to the filter.

The proximal plug may define a cylinder axis. Prior to the delivery, the cylinder axis may be disposed coaxially with the longitudinal axis. The cylinder axis may be disposed coaxially with the longitudinal axis when the plug and collar are sealed together prior to initiation of the urging of the fluid toward the distal outlet. Such coaxial dispositions may be approximately coaxial.

The proximal plug may include one or more than one tab extending radially outward orthogonal the cylinder axis. The tab(s) may be disposed distal to the plug groove. The tab(s) may be disposed at a distal end of the plug. An effective cross-sectional diameter of the plug in a plane of the tab(s) may be about 50% to about 95% of an internal diameter of the inner wall of the filter cap cylindrical body.

The tab(s) may be configured to maintain an orientation of the cylinder axis coaxial with the longitudinal axis during distal displacement of the plug relative to the collar. The tab(s) may be configured to maintain the orientation of the cylinder axis coaxial with the longitudinal axis after the distal displacement. The tab(s) may maintain the orientation of the cylinder axis coaxial with the longitudinal axis by interference between the tabs and the internal wall of the filter cap cylindrical body. The tab(s) may maintain the orientation of the cylinder axis coaxial with the longitudinal axis by interference between the internal wall and radially extreme edges of the tabs. Such coaxial orientations may be approximately coaxial.

The plug may include one or more than one inter-tab gap. The inter-tab gap(s) may be disposed along an exterior surface of the plug. The inter-tab gap(s) may be disposed between circumferentially adjacent facing lateral edges of the tab(s). In embodiments featuring two or more tabs, the inter-tab gap(s) may be disposed between circumferentially adjacent facing lateral edges of circumferentially adjacent tabs. The inter-tab gap(s) may be configured to allow the flow of the fluid between the proximal exterior of the filter housing and the interior of the cylindrical body during the distal displacement of the plug relative to the collar into the interior of the cylindrical body. The inter-tab gap(s) may be configured to allow the flow of the fluid between the proximal exterior of the filter housing and the interior of the cylindrical body after the distal displacement of the plug. During and after the distal displacement, the inter-tab gap(s) may provide passage for the fluid flowing, from the proximal exterior of the fluid housing into the interior of the cylindrical body, to the filter.

In another embodiment of the filter cap, the cylindrical body may include a diaphragm ring support disposed at a proximal end of the body. The ring support may be disposed orthogonal the longitudinal axis. Such orthogonal disposition may be approximate. The ring support may have a circumference concentric with the cylindrical body. The ring support may support a diaphragm disposed within the circumference of the ring support. The ring support may support a diaphragm concentric with the circumference of the ring support. The diaphragm may occupy a region concentric with the circumference of the ring support. The diaphragm may fully occupy the region concentric with the circumference. The region may have an area spanning about 30% to about 95% of the proximal exterior side of the filter housing. The region may lie proximal to the ring support. The region may lie at least approximately planar with the ring support. The region may lie distal to the ring support.

The filter cap may include the diaphragm. The diaphragm, prior to the urging, may seal the proximal side of the filter housing against the flow of the fluid from the exterior of the proximal exterior of the fluid housing into the interior of the filter cap. Prior to the urging, the diaphragm may close the proximal side of the filter housing, preventing the flow into the interior of the filter cap. Attendant upon the urging, the diaphragm may open the proximal side of the filter housing to the flow into the interior of the filter cap.

The diaphragm may include an initially closed diaphragm. The initially closed diaphragm may prevent the flow of the fluid to the filter. The diaphragm may be configured to be opened by the distally urged fluid. The diaphragm, when open, may be configured to provide passage to the flow of the fluid to the filter.

The diaphragm may include a slit diaphragm. The slit diaphragm may include one or more than one slit. The slit diaphragm may include a plurality of slits. Prior to the initiation of the distally directed urging and its attendant increase of the fluid pressure on the proximal side of the distal cap, the slit(s) may not fully penetrate a thickness of the diaphragm. The increase of the fluid pressure may fully open the slit(s) across the thickness of the diaphragm. Prior to the initiation of the distally directed urging and its attendant increase of the fluid pressure on the proximal side of the distal cap, the slit(s) may not fully penetrate the thickness of the diaphragm along a full length of the slit(s). The increase of the fluid pressure may more fully open the slit(s) along the full length of the slit(s).

Prior to the initiation of the distally directed urging and its attendant increase of the fluid pressure on the proximal side of the distal cap, the slit(s) may fully penetrate the thickness of the diaphragm. Parallel facing edges of the slit(s) may compress against each other, prior to the initiation of the urging, to provide a seal against the flow of the fluid from the proximal exterior of the filter cap into the interior of the filter cap.

The diaphragm may include one or more than one diaphragm flap. The diaphragm flap(s) may close along facing edges of the slit(s). The diaphragm flap(s), when closed, may present an area coextensive with the diaphragm. The diaphragm flap(s), when opened by the increase of the fluid pressure upon the closed proximal exterior side of the filter housing, may extend distally into the filter cap. Portions of the diaphragm flap(s), when the diaphragm flap(s) are opened, may be disposed within the interior of the cylindrical body. Portions of the opened diaphragm flaps may be disposed within the interior of the cylindrical body at an acute angle from closed positions of the diaphragm flaps. Portions of the opened diaphragm flaps may be disposed approximately parallel to the inner wall of the cylindrical body. Portions of the opened diaphragm flaps may be disposed within the interior of the cylindrical body at an obtuse angle from closed positions of the diaphragm flaps.

The diaphragm flap(s), when opened, may fold distally into the filter cap. The diaphragm flap(s) may fold at hinge elements supported by the diaphragm ring support. The hinge elements may include regions along a distal face of the diaphragm. The regions along the distal face may include grooves extending into a material of the diaphragm. The grooves may extend proximally into the material of the diaphragm from the distal surface and/or may extend distally into the material from the proximal surface. The grooves extending into the material of the diaphragm may be disposed along line segments encompassed by the ring support. The grooves extending into the material of the diaphragm may be disposed along arcs concentric with the cylindrical body. The material of the diaphragm along the grooves may be more likely to fold than the material of the diaphragm disposed away from the grooves. The material of the diaphragm along the grooves may be more likely to fold distally than the material of the diaphragm disposed away from the grooves.

The diaphragm may be configured to re-close after being opened. The diaphragm may be configured to re-close after cessation of the flow of the fluid. The diaphragm may be configured to re-close after cessation of the distally directed urging of the fluid.

The apparatus may include a filter housing for embedment within a medicament container of a device for delivering filtered medicament. The device may define a longitudinal axis. The device may deliver the filtered medicament from a distal outlet of the device. The filter housing may be configured to be embedded in the container proximal to the distal outlet.

The filter housing may include a filter base. The filter base may be configured to support a filter. The filter may be disposed on the filter base.

The filter may include acrylic polymer. The filter may include polyethersulfone. The filter may include any suitable material. Any suitable material may include polyvinylidene fluoride.

The filter may be configured to prevent passage through the filter of particles larger than a largest pore size of the filter. The filter may be configured to retain particles larger than the largest pore size of the filter. The filter may be configured to retard passage through the filter of particles larger than an average pore size of the filter. The filter may be configured to retain particles larger than the average pore size of the filter.

The average pore size of the filter may be about five microns. The average pore size of the filter may be any suitable average pore size. Any suitable average pore size may about 0.2 microns.

The filter housing may be configured to provide a seal of a perimeter of the filter. When sealed, the perimeter may be configured to prevent passage of the medicament around the perimeter.

An exterior of the filter housing may be configured to provide a seal against an inner wall of the container. The seal may be configured to prevent distal flow of the medicament between the inner wall and the filter housing.

The medicament may be disposed within the container. The medicament may be disposed within the container proximal the filter. The medicament may be disposed within the container distal to a longitudinally slidable plunger. The plunger may be configured to seal against the inner wall. The plunger may be configured to seal slidingly against the inner wall. The plunger may be configured to urge the medicament toward the distal outlet.

The filter housing may include a filter cap. The filter cap may be configured to join with the base.

The medicament may be in contact with a proximal exterior aspect of the filter cap. Prior to the delivery of the medicament, the filter cap may be configured to block access to the filter by the medicament. Prior to the delivery of the medicament, the proximal exterior aspect of the filter cap may be configured to block access to the filter by the medicament.

The filter cap may be configured to be unblocked by distal urging of the medicament upon the proximal exterior aspect of the filter cap. The unblocked filter cap may allow flow of the medicament to the filter.

The proximal exterior aspect of the filter cap may include a proximal plug disposed in a collar of the filter cap. The proximal plug may seal the collar. The proximal plug may seal the collar against flow of the medicament. The proximal plug may seal the collar against flow of the medicament into an interior of the proximal cap. The proximal plug may seal the collar against flow of the medicament into an interior of the filter housing toward the filter. The proximal plug may be configured to be distally displaced, relative to the collar, by distally urged medicament. Displacing the proximal plug relative to the collar may unseal the collar to the flow of the medicament through the collar.

The proximal exterior aspect of the filter cap may include a diaphragm. The diaphragm may be a slit diaphragm. The diaphragm may be initially closed. Prior to the delivery of the medicament, the diaphragm may be closed. Prior to the delivery of the medicament, the diaphragm may be closed against flow of the medicament. The diaphragm may be configured to be opened by distally urged medicament. The opened diaphragm may provide passage to the flow of the medicament.

The methods of manufacture of the apparatus may include a method of manufacturing a medicament device for delivery of a filtered medicament from a distal outlet of the delivery device. The device may define a longitudinal axis.

The method of manufacturing the device may include providing a medicament container disposed coaxially with the axis. The container may have a distal outlet. The container may have a proximal opening.

The method may include assembling a filter housing. Assembling the filter housing may include providing a filter base. The filter base may be configured to support a filter. Assembling the filter housing may include disposing the filter upon a face of the base. The face may be an exterior aspect The face may be an upper face of the base.

Assembling the filter housing may include sealing a perimeter of the filter against fluid flow. The filter housing may include an interior surface configured to seal the perimeter of the filter against fluid flow. The interior surface may be a surface of the base. The interior surface may be a circumferential surface of the base. The interior surface may be the face of the base. The filter may be sealed to the interior surface via a laser welding process. The filter may be sealed to the interior surface via any suitable process. Any suitable process may include depolymerization and subsequent repolymerization of a material of the interior surface and/or of the filter against each other.

Assembling the filter housing may include providing a filter cap. Assembling the filter housing may include joining the base with the filter cap. The cap may include a rib. The rib may be disposed on an exterior of the cap. The rib may be disposed on an exterior circumference of the cap. The rib may encircle the exterior circumference of the cap. The rib may be configured to seal against an inner wall of the container.

The cap may include an exterior aspect. The exterior aspect may include a structural component of the cap. The exterior aspect may be configured to block fluid access to the filter before the delivery. The exterior aspect may be configured to be unblocked to provide fluid access to the filter during the delivery.

The method may include introducing the filter housing into the container. The filter housing may be introduced into the container through the proximal opening. The filter housing may be introduced into the container with the base disposed distally. The filter housing may be introduced into the container with the base disposed distally relative to the axis.

The method may include embedding the filter housing in the container. The filter housing may be embedded in the container proximal the distal outlet.

The method may include sealing the rib against the inner wall. Sealing the rib against the inner wall may provide a fluid-tight seal against flow of medicament between the exterior of the cap and the inner wall. Sealing the rib against the inner wall may provide a fluid-tight seal against flow of medicament between an exterior of the filter housing and the inner wall.

The method may include transferring the medicament to the container via the proximal opening.

The method may include providing a longitudinally slidable plunger. The method may include inserting the plunger into the container via the proximal opening. The plunger may be configured to seal the medicament within the container. The plunger may be configured to slidingly seal against the inner wall. The plunger may be configured to urge the medicament toward the distal outlet. The inserting may be performed without distally urging the medicament sufficiently to unblock the exterior aspect.

The method may include providing a plunger rod. The method may include introducing the plunger rod into the container via the proximal opening. The plunger rod may be configured to urge the plunger distally within the container. The plunger rod may be configured to abut a proximal face of the plunger. The plunger rod may be configured to attach to the proximal face of the plunger.

The filter housing may be introduced into the container with the exterior aspect disposed proximally relative to the axis. The exterior aspect may include an initially closed diaphragm configured to be opened by distally urged medicament; and, thereby, provide passage to flow of the medicament. The exterior aspect may include a proximal plug disposed in, and sealing, a proximal collar of the filter cap. The plug may be configured to be distally displaced, relative to the collar, by distally urged medicament; and, thereby, unseal the collar to a flow of the medicament therethrough.

The medicament may include a formulation of one or more compounds. The compounds may include naturally occurring substances. The compounds may include substances derived from naturally occurring substances. The compounds may include synthetically produced substances. The compounds may include chimeric substances. The compounds may include engineered substances. The compounds may include humanized substances. The compounds may include substances produced by recombinant techniques. The compounds may include substances modified by recombinant techniques.

The compounds may include a drug accepted for therapeutic patient treatment. The compounds may include a substance used in a therapeutic protocol. The compounds may include a substance used in a diagnostic protocol. The compounds may include a substance used in an experimental protocol. The compounds may include a substance compatible for use with apparatus and methods of the invention.

The medicament may include any medical agent listed herein, either alone or in combination with one or more other listed medical agents or with one or more other, non-listed, medical agents. The medical agents may include anti-glaucoma medications, other ocular agents, neuroprotective agents, antimicrobial agents, anti-inflammatory agents (including steroids and non-steroidal compounds), and biological agents including hormones, enzymes or enzyme-related components, antibodies or antibody-related components, oligonucleotides (including DNA, RNA, short-interfering RNA, and other suitable oligonucleotides, such as antisense oligonucleotides), DNA/RNA vectors, viruses or viral vectors, peptides, and proteins. The medical agents may include anti-angiogenesis agents, including angiostatin, anecortave acetate, thrombospondin, vascular endothelial growth factor (VEGF) receptor tyrosine kinase inhibitors, and anti-VEGF drugs, such as ranibizumab (LUCENTIS®), bevacizumab (AVASTIN®), pegaptanib (MACUGEN®), sunitinib, and sorafenib, and any of a variety of known small-molecule and transcription inhibitors having an anti-angiogenesis effect; ophthalmic drugs, including glaucoma agents, such as adrenergic antagonists, including beta-blocker agents such as atenolol, propranolol, metipranolol, betaxolol, carteolol, levobetaxolol, levobunolol and timolol. The medical agents may include platelet-derived growth factor (PDGF) inhibitors and anti-PDGF drugs. The medical agents may include transformation growth factor (TGF) inhibitors and anti-TGF drugs. The medical agents may include anti-inflammatory agents including glucocorticoids and corticosteroids, such as betamethasone, cortisone, dexamethasone, dexamethasone 21-phosphate, methylprednisolone, prednisolone 21-phosphate, prednisolone acetate, prednisolone, loteprednol, medrysone, fluocinolone acetonide, triamcinolone acetonide, triamcinolone, beclomethasone, budesonide, flunisolide, fluorometholone, fluticasone, hydrocortisone, hydrocortisone acetate and rimexolone; and non-steroidal anti-inflammatory agents including diclofenac, flurbiprofen, ibuprofen, bromfenac, nepafenac, ketorolac, salicylate, indomethacin, naxopren, naproxen, piroxicam and nabumetone. The medical agents may include anti-cytokine agents; the medical agents may include anti-interleukin-6 agents such as tocilizumab (ACTEMRA®). The medical agents may include anti-complement agents, including those targeting complement factor D (such as an anti-complement factor D antibody or an antigen-binding fragment thereof) such as lampalizumab, and those targeting complement factor H (such as an anti-complement factor H antibody or an antigen-binding fragment thereof). The medical agents may include angiopoietin-specific agents, such as an angiopoietin-2 antibody or an antigen-binding fragment thereof. The medical agents may include human growth hormone. The medical agents may include any suitable medical agent, whether or not listed above.

The medicament may include one or more derivatives of any of the above-mentioned medical agents. The medicament may include advanced forms of any of the above-mentioned medical agents. The medicament may include mutated forms of any of the above-mentioned medical agents. The medicament may include combinations of any of the above-mentioned medical agents. The combinations may be incorporated into a multi-specific molecule. The multi-specific molecule may exhibit properties of its constituent parts. The multi-specific molecule may exhibit properties different from any if its constituent parts.

The volume of medicament enclosed in the device may be substantially determined during manufacture. The volume may be set with anticipation of loss of medicament that may result from manipulations of the device prior to performance of a medicament-delivery stroke via a final distal displacement of the plunger within the syringe barrel toward the filter. Such manipulations may include syringe priming. The volume may depend on the scenario for which the device is intended. Illustrative ranges of value of the volume may include about 0.025 milliliter to about 0.05 milliliter, about 0.05 milliliter to about 0.1 milliliter, about 0.1 milliliter to about 0.25 milliliter, about 0.25 milliliter to about 0.5 milliliter, about 0.5 milliliter to about 1 milliliter, about 1 milliliter to about 2 milliliters, about 2 milliliters to about 3 milliliters, about 3 milliliters to about 4 milliliters, about 4 milliliters to about 5 milliliters, about 5 milliliters to about 6 milliliters, about 6 milliliters to about 7 milliliters, about 7 milliliters to about 8 milliliters, about 8 milliliters to about 9 milliliters, about 9 milliliters to about 10 milliliters, or any other suitable range of volumes. Any other suitable range of volumes may be enclosed in the device.

Providing the filter base may include manufacturing the filter base. Providing the filter cap may include manufacturing the filter cap. Manufacturing the filter base and/or the filter cap may be accomplished through molding processes. Manufacture of the filter base and/or the filter may be accomplished through injection molding processes. The injection molding processes may include double injection molding processes.

Providing the plunger may include manufacturing the plunger. Providing the plunger rod may include manufacturing the plunger rod. Manufacturing the plunger and/or the plunger rod may be accomplished through molding processes. Manufacture of the plunger and/or of the plunger rod may be accomplished through injection molding processes. The injection molding processes may include double injection molding processes.

The method may involve election of materials for manufacture of components of the delivery. The materials may be elected for their material properties. The material properties for which the materials may be elected may facilitate operation of the apparatus. The material properties for which the materials may be elected may facilitate operation of the delivery device. The properties may include chemical inertness, resilience, transparency and other suitable properties. Other suitable properties may include hardness.

For instance, for the filter, the properties may include average pore size, resilience against impact stress, and other relevant properties. Other relevant properties may include chemical inertness. Other relevant properties may include sealability against material of the filter housing.

The filter may include a filter material. The filter material may include polymeric material. The filter material may include acrylic polymer. The filter material may include polyethersulfone. The filter material may include any suitable filter material. Any suitable filter material may include polyvinylidene fluoride.

The plunger may include a plunger material. The plunger material may include polymeric material. The plunger material may include elastomeric material. The plunger material may include thermoplastic elastomer (TPE). The plunger material may include natural rubber. The plunger material may include a compound made from natural rubber. The plunger material may include synthetic rubber. The plunger material may include a compound made from synthetic rubber. The plunger material may include silicone rubber.

The plunger material may include a compound made from silicone rubber. The plunger material may include butyl rubber. The plunger material may include a compound made from butyl rubber. The plunger material may include a material elected to reduce interaction of the plunger with the medicament. The plunger material may include a resilient material. The plunger material may include a material with a hardness less than about 80 Shore A durometer (ASTM D2240 type A hardness scale). The plunger material may facilitate movement of the plunger within the barrel. The plunger material may facilitate engagement of the plunger with the inner wall of the barrel. The plunger material may facilitate sealing of the plunger against the inner wall of the barrel.

The plunger material may include a plunger lubricious material. The plunger lubricious material may coat the plunger material. The plunger material may bear the plunger lubricious material. The plunger lubricious material may include polytetrafluoroethylene (PTFE). The plunger lubricious material may include ethylene tetrafluoroethylene (ETFE). The plunger lubricious material may include silicone oil. The silicone oil may be cross-linked silicone oil. The plunger lubricious coating may include a material elected to reduce interaction of the plunger with the medicament. The plunger lubricious material may facilitate movement of the plunger within the barrel. The plunger lubricious material may facilitate engagement of the plunger with the inner wall of the barrel. The plunger lubricious material may facilitate sealing of the plunger against the inner wall of the barrel.

The plunger rod may include a rod material. The rod material may include polymeric material. The rod material may include thermoplastic polymer. The rod material may include polyoxymethylene. The rod material may include polypropylene. The rod material may include nylon. The rod material may include PTFE. The rod material may include ABS. The rod material may include polycarbonate. The rod material may include polysulfone. The rod material may include acrylic polymer. The rod material may include poly (methyl methacrylate) (PMMA). The rod material may include a stiff material. The rod material may include a material with a hardness more than about 80 Shore A durometer. The rod material may include a material with a low coefficient of friction. The rod material may include a material with a coefficient of friction of about 0.01 to about 0.5.

The rod material may include a rod lubricious material. The rod lubricious material may coat the rod material. The rod material may bear the rod lubricious material. The rod lubricious material may include one or more of the plunger lubricious materials. For instance, the rod lubricious material may include silicone oil. The silicone oil may be cross-linked silicone oil. The rod lubricious material may facilitate movement of the plunger rod within the container proximal opening.

The container may include a barrel material. The barrel material may include polymeric material. The barrel material may include amorphous material. The barrel material may include a polymer. The barrel material may include thermoplastic polymer. The barrel material may include cyclic olefin polymer (COP). The barrel material may include cyclic olefin copolymer (COC). The barrel material may include polypropylene. The barrel material may include PMMA. The barrel material may include polycarbonate. The barrel material may include glass. The barrel material may include type 1 borosilicate glass. The barrel material may include a material that is translucent. The barrel material may include a material that is transparent.

The barrel material may include a barrel lubricious material. The barrel lubricious material may coat the barrel material. The barrel lubricious material may coat the inner wall of the container. The barrel material may bear the barrel lubricious material. The barrel lubricious material may include one or more of the plunger lubricious materials. For instance, the barrel lubricious material may include silicone oil. The silicone oil may be cross-linked silicone oil. The barrel lubricious material may facilitate movement of the plunger within the barrel.

The filter base may include a filter base material. The base material may include polymeric material. The base material may include thermoplastic elastomer (TPE). The base material may include one or more of the plunger materials. The base material may include material harder than the plunger material.

The filter cap may include a filter cap material. The cap material may include polymeric material. The cap material may include thermoplastic elastomer (TPE). The cap material may include one or more of the plunger materials. The cap material may include material harder than the plunger material. The cap material may include material less hard than the plunger material.

The cap material may include diaphragm material. The cap material may include plug material. The plug material and/or the diaphragm material may include material harder than the plunger material. The plug material and/or the diaphragm material may include material less hard than the plunger material.

Apparatus and methods described herein are illustrative. Apparatus and methods in accordance with the invention will now be described in connection with the FIGs. The FIGs. show illustrative features of apparatus in accordance with the principles of the invention.

Some apparatus may omit features shown and/or described in connection with illustrative apparatus. Some embodiments may include features that are neither shown nor described in connection with the illustrative methods. Features of illustrative apparatus may be combined. For example, one illustrative embodiment may include features shown in connection with another illustrative embodiment.

Apparatus may involve some or all of the features of the illustrative apparatus and/or some or all of the steps of the illustrative methods.

The apparatus and methods of the invention will be described in connection with embodiments and features of illustrative devices. The devices will be described now with reference to the accompanying drawings in the FIGs., which form a part hereof. It is to be understood that other embodiments may be utilized and that structural, functional and procedural modifications may be made without departing from the scope and spirit of the present invention.

The FIGs. employ like part numbers, in the tens and units places, to refer to like features.

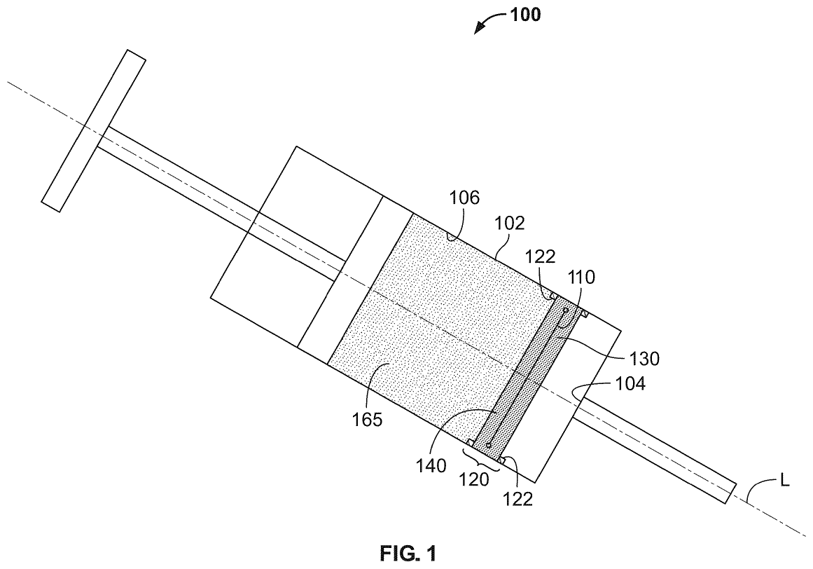

FIG. 1 shows, in simplified form, illustrative fluid delivery device 100. Delivery device 100 may define longitudinal device axis L. (Longitudinal axis L is understood to be defined in all subsequent FIGs., even if not shown.) Delivery device 100 may include container 102. Container 102 may be disposed coaxially with axis L. Container 102 may include a syringe barrel. Container 102 may include inner barrel wall 106. Container 102 may contain fluid 165. Container 102 may be pre-filled with fluid 165. Fluid 165 may include a medicament.

Filter housing 120 may be lodged in container 102. Filter housing 120 may be lodged in container 102 proximal to distal outlet 104. Filter housing 120 may be disposed orthogonal to axis L. Filter housing 120 may include embedment elements 122. Embedment elements 122 may facilitate lodging of filter housing 120 within container 102.

The lodging of filter housing 120 in container 102 may be fluid-tight. Embedment elements 122 may compress against inner wall 106. Compression of embedment elements 122 against inner wall 106 may prevent passage of fluid 165 between inner wall 106 and a longitudinal exterior of filter housing 120.

Filter housing 120 may include distal filter base 130. Filter housing 120 may include proximal filter cap 140. Filter base 130 and filter cap 140 may be secured to each other. Filter base 130 and filter cap 140 may be sealed to each other.

Filter housing 120 may include filter 110. Filter housing 120 may support filter 110. Filter 110 may be affixed within filter housing 120. Filter 110 may be sealed into filter housing 120. Filter housing 120 may maintain filter 110 athwart axis L.

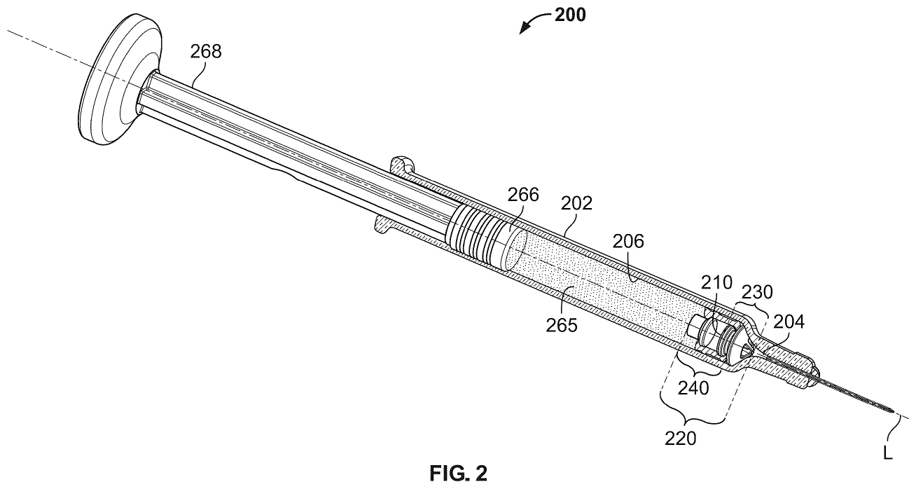

FIG. 2 shows illustrative fluid delivery device 200. Delivery device 200 may include apparatus features with the same relationships, properties and functions as features of device 100 (shown in FIG. 1), above-described, as indicated by like reference characters. These apparatus features may include axis L, container 202, distal outlet 204, inner wall 206, filter 210, filter housing 220, filter base 230, filter cap 240 and fluid 265 (depicted illustratively as speckles).

Device 200 may include syringe plunger rod 268. Plunger rod 268 may abut syringe plunger 266. Plunger rod 268 may be connected to plunger 266.

As depicted, fluid 265 may be bordered by plunger 266, inner wall 206 and filter housing 220. More specifically, fluid 265 may be bordered by a distal face of plunger 266; by inner wall 206; and by proximal exterior aspects of filter cap 240.

Plunger rod 268 may be configured to move plunger 266 distally within container 202. Plunger 266 may be configured to urge fluid 265 toward distal outlet 204. Distally urged fluid 265 may be configured to apply distally directed force to proximal exterior aspects of filter cap 240.

Filter 210 is shown just proximal to filter base 230. A region proximal to filter 210 and distal to the proximal exterior aspects of filter cap 240 bordering fluid 265, is depicted as devoid of fluid 265. Similarly, outlet 204 is depicted as devoid of fluid. In general, hollow device regions disposed distal to proximal exterior aspects of filter cap 240 are depicted as devoid of fluid in FIGs. illustrating the invention in operational configurations before initiation of urging of fluid 265 toward distal outlet 204. Such operational configurations may be deemed pre-filtering configurations, inclusive of PFS storage configurations.

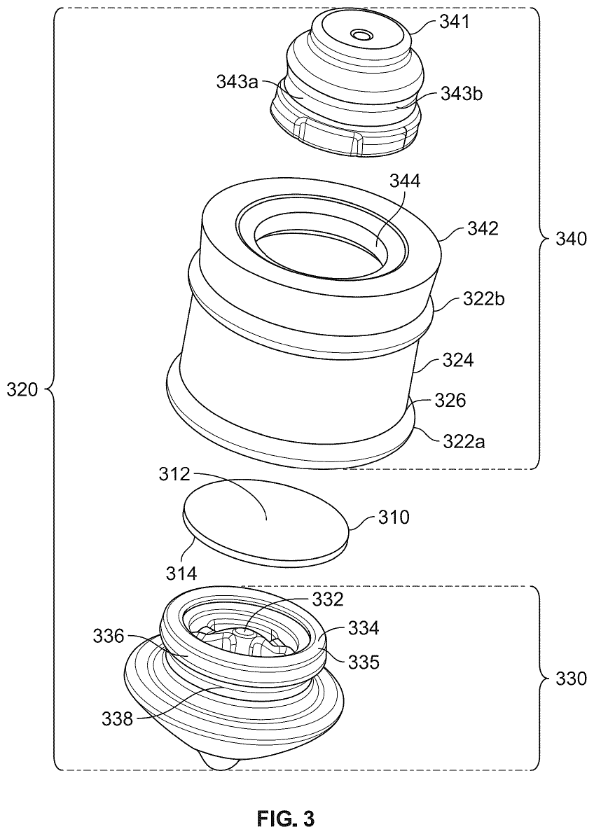

FIG. 3 shows details of apparatus features that may have the Same relationships, properties and functions as features of one or both of delivery devices 100 and 200 (shown in FIGS. 1 and 2, respectively), above-described, as indicated by like reference characters. These apparatus features may include filter 310, filter housing 320, embedment elements 322a and 322b (collectively, embedment elements 322), filter base 330 and filter cap 340.

The exploded view of FIG. 3 depicts filter base 330 and filter cap 340 separate from each other, with filter 310 spaced in-between. FIG. 3 also depicts filter cap proximal plug 341 and filter cap proximal collar 342 separate from each other. FIG. 3 may afford unobstructed views of features of filter housing 320 that may be more difficult to discern in views of assembled filter housing 320 (such as filter housing 220, shown in FIG. 2).

Filter 310 may include filter central area 312. Filter 310 may include filter periphery 314. Periphery 314 may be a distal periphery. Periphery 314 may extend radially inward from a perimeter of filter 310.

Filter base 330 may include proximal central projection 332. Central projection 332 may be configured to provide support to filter 310. Central projection 332 may be configured to provide support to central area 312.

Filter base 330 may include proximal rim 335. Proximal rim 335 may support proximal rim surface 334. Proximal rim 335 may be configured to provide support to filter 310. Proximal rim 335 may be configured to provide support to periphery 314. Periphery 314 may be disposed upon surface 334. Periphery 314 may be sealed to surface 334. All of periphery 314 may be sealed to surface 334. Periphery 314 may be sealed to surface 334 along all of surface 334. Sealing periphery 314 along surface 334 may provide a fluid-tight seal configured to prevent passage of fluid between an interior of filter base 330 and any of the perimeter of filter 310.

Filter base 330 may include circumferential convexity 336. Filter base 330 may include circumferential concavity 338. Convexity 336 and/or concavity 338 may be configured to mechanically interfere with distal interior features (not shown) of filter cap 340. Interference of convexity 336 and/or concavity 338 with the interior features of filter cap 340 may fit together filter base 330 and filter cap 340. Interference of convexity 336 and/or concavity 338 with the interior features of filter cap 340 may secure together filter base 330 and filter cap 340.

Convexity 336 and/or concavity 338 may be configured to compress against the interior features of filter cap 340. Compression of convexity 336 and/or concavity 338 against the interior features of filter cap 340 may provide a fluid-tight seal between filter base 330 and filter cap 340. The interior features of filter cap 340 may be configured to compress against convexity 336 and/or concavity 338. Compression of the interior features of filter cap 340 against convexity 336 and/or concavity 338 may provide a fluid-tight seal between filter base 330 and filter cap 340.

Filter cap 340 may include hollow cylindrical body 324. Cylindrical body 324 may include distal rim 326. Distal rim 326 may be configured to receive at least portions of filter base 330. Distal rim 326 may be configured to longitudinally receive filter 310 and proximal rim 335.

Cylindrical body 324 may include distal embedment element 322a. Embedment element 322a may lie along distal rim 326. Embedment element 322a may lie along a circumferential exterior of cylindrical body 324. Cylindrical body 324 may include proximal embedment element 322b. Embedment element 322b may lie along a circumferential exterior of cylindrical body 324.

Each of embedment elements 322 may be configured to compress against the inner barrel wall of the delivery device (such as against inner wall 106 of delivery device 100, shown in FIG. 1). Compression of embedment elements 322 against the inner barrel wall may provide a fluid-tight seal between the inner barrel wall and each of embedment elements 322.

Proximal plug 341 may include plug groove 343a. Groove 343a may extend radially inward into a body of plug 341. Groove 343a may extend circumferentially around the body. Proximal plug 341 may include proximal groove rise 343b. Groove 343a may include groove rise 343b. Groove rise 343b may extend circumferentially around the body. Groove rise 343b may include a proximal side of groove 343a. Groove rise 343b may extend less deeply into the body than a full depth of groove 343a.

Proximal collar 342 may include inwardly protruding proximal rim 344. Rim 344 may be configured to mechanically interfere with groove 343a. Interference of groove 343a and rim 344 may fit together plug 341 and collar 342. The interference of groove 343a and rim 344 may secure together plug 341 and collar 342. Groove rise 343b may be configured to maintain plug 341 within collar 342, providing contact/frictional forces by interfering with rim 344 to oppose distally directed forces that may act on a proximal exterior of plug 341.

Rim 344 may be configured to compress against groove 343a. Compression of rim 344 against groove 343a may provide a fluid-tight seal between plug 341 and collar 342, thereby sealing assembled filter cap 340 against fluid in a pre-filtering configuration of the device.

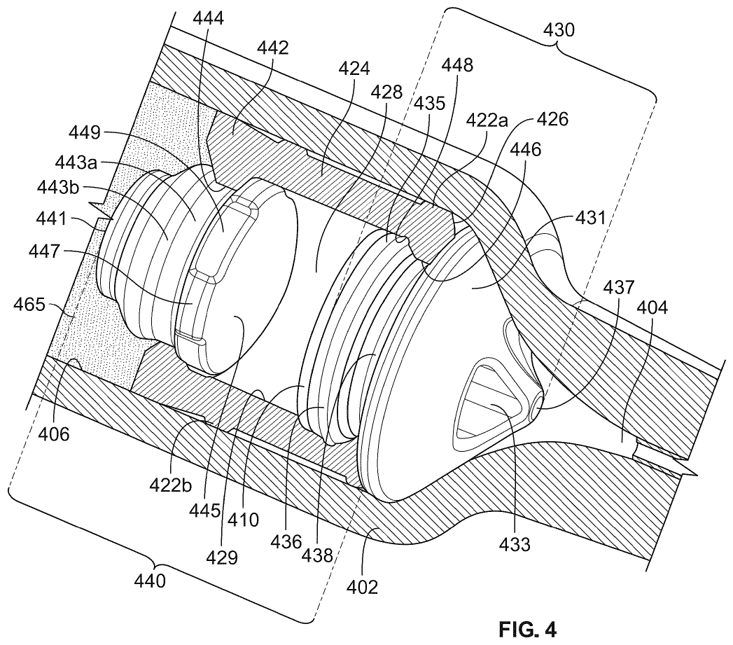

FIG. 4 shows details of apparatus features that may have the same relationships, properties and functions as features of one or both of delivery devices 100 and 200 (shown in FIGS. 1 and 2, respectively) and/or be shown in FIG. 3, above-described, as indicated by like reference characters. These apparatus features may include container 402, distal outlet 404, inner wall 406; filter 410; embedment elements 422, cylindrical body 424, rim 426; filter base 430, rim 435, convexity 436, concavity 438; filter cap 440, plug 441, collar 442, groove 443a, groove rise 443b, inwardly protruding rim 444; and fluid 465.

FIG. 4 depicts cylindrical body 424 lodged within container 402, with embedment elements 422a and 422b compressed against inner wall 406. Compression of embedment elements 422 against inner wall 406 may provide fluid-tight sealing against passage of fluid 465 between inner wall 406 and a longitudinal exterior of cylindrical body 424.

Inwardly protruding proximal rim 444 is shown interfering with plug groove 443a and plug groove rise 443b. Interference of rim 444 with groove 443a and groove rise 443b may fit together plug 441 and collar 442. The interference of rim 444 with groove 443a and groove rise 443b may secure together plug 441 and collar 442.

The interference of rim 444 with groove 443a and/or with groove rise 443b may provide contact/frictional forces that may maintain plug 441 within collar 442. The contact/frictional forces may be configured to oppose distally directed force that may be applied to a proximal exterior of plug 441 by distally urged fluid 465.

An increase of pressure applied to fluid 465 may result in an increase in magnitude of distally directed force applied to the proximal exterior of plug 441 by the distally urged fluid 465. The increase in magnitude of the distally directed forces applied to the proximal exterior of plug 441 by the distally urged fluid 465, may overcome the contact/frictional forces maintaining plug 441 within collar 442.

Rim 444 may be configured to compress against groove 443a. Rim 444 may be configured to compress against groove rise 443b. Compression of rim 444 against groove 443a and/or against groove rise 443b may provide a fluid-tight seal between plug 441 and collar 442. The seal between plug 441 and collar 442 may prevent passage of fluid 465 between plug 441 and collar 442. The seal between plug 441 and collar 442 may prevent passage of fluid 465 into interior 428 of cylindrical body 424.

Materials and/or geometries of collar 442 (in particular, of rim 444) and/or of plug 441 (in particular, of groove 443a and groove rise 443b) may be pre-selected to maintain plug 441 sealed within collar 444 (as shown) under pre-filtering pressures of fluid 465; and (not shown), to have plug 441 distally displaced relative to rim 444 into interior 428, under higher pressures readily achievable by the user during initiation of the urging of fluid 465 toward distal outlet 404, opening collar 442 to flow of fluid 465 through rim 444.

Filter cap proximal plug 441 may include distal plug face 445. Plug face 445 may be supported by a distal section of a body of plug 441. The distal section of the body of plug 441 may extend radially outward as tab(s) 447. Tab(s) 447 may extend orthogonal to a cylinder axis (not shown) defined by plug 441. The cylinder axis may intersect a center of face 445. The cylinder axis may intersect a center of a proximal exterior face of plug 441.

Plug 441 may be maintained within collar 444 (as shown) with an orientation of the cylinder axis at least approximately coaxial with longitudinal axis L (not shown). When plug 441 is displaced into interior 428 upon initiation of the urging of fluid 465 toward distal outlet 404, tab(s) 447 may maintain the orientation of the cylinder axis approximately coaxial with the longitudinal axis. During and after distal displacement of plug 441, radially extreme edges of tab(s) 447 may interfere with internal wall 429 of cylindrical body 424, maintaining the cylinder axis approximately coaxial with longitudinal axis L. Interference of the radially extreme edges of tab(s) 447 with internal wall 429 may occur intermittently as plug 441 wobbles in its longitudinal transit toward filter 410 and, thereafter, as fluid 465 continues to flow distally past plug 441 toward filter 410. Such intermittent interference may preclude plug 441 from becoming skewed against and possibly wedged between sides of internal wall 429.

Filter cap proximal plug 441 may include inter-tab gap(s) 449 disposed, orthogonal to the cylinder axis, between circumferentially adjacent facing lateral edges of tab(s) 447. During and after the distal displacement of plug 441, inter-tab gap(s) 449 may provide passage for distal flow of fluid 465 past plug 441 toward filter 410.

During and after the distal displacement of plug 441, the distal flow of fluid 465 past plug 441 via inter-tab gap(s) 449 (as well as distal flow of fluid 465 between internal wall 429 and the radially extreme edges of tab(s) 447) may provide cushioning between plug 441 and internal wall 429. Flow of fluid 465 across face 445 as face 445 approaches filter 410 during distal displacement of plug 441, may provide cushioning between plug 431 and filter 410. The flow of fluid 465 across face 445 when face 445 resides closely proximal to filter 410 after distal displacement of plug 441, may provide cushioning between plug 431 and filter 410.

Filter cap distal rim 426 may extend radially outward as embedment element 422a. Rim 426 may extend radially inward as filter cap convexity 446. Filter cap convexity 446 may lie along rim 426. Filter cap convexity 446 may lie along a circumferential interior of cylindrical body 424. Filter cap concavity 448 may be disposed parallel to filter cap convexity 446. Filter cap concavity 448 may lie along a circumferential interior of cylindrical body 424.

Filter cap convexity 446 may mechanically interfere with filter base concavity 438. Interference of filter cap convexity 446 and filter base concavity 438 may fit together filter cap 440 and filter base 430. The interference of filter cap convexity 446 and filter base concavity 438 may secure together filter cap 440 and filter base 430. Filter cap convexity 446 may be configured to compress against filter base concavity 438. Compression of filter cap convexity 446 against filter base concavity 438 may provide a fluid-tight seal between filter cap 440 and filter base 430. The seal between filter cap 440 and filter base 430 may prevent passage of fluid 465 between filter cap convexity 446 and filter base concavity 438.

Filter base convexity 436 may mechanically interfere with filter cap concavity 448. Interference of filter base convexity 436 and filter cap concavity 448 may fit together filter cap 440 and filter base 430. The interference of filter base convexity 436 and filter cap concavity 448 may secure together filter cap 440 and filter base 430. Filter base convexity 436 may be configured to compress against filter cap concavity 448. Compression of filter base convexity 436 against filter cap concavity 448 may provide a fluid-tight seal between filter cap 440 and filter base 430. The seal between filter cap 440 and filter base 430 may prevent passage of fluid 465 between filter base convexity 436 and filter cap concavity 448.

Distal to the seal(s) between filter base 430 and filter cap 440, filter base 430 may include (frusto) conical surface 431. Surface 431 may be contoured complementarily to a distal internal taper of container 402 leading into distal outlet 404. Surface 431 may compress against the distal internal taper. Compression of surface 431 against the distal internal taper may provide a fluid-tight seal between surface 431 and the distal internal taper. The seal between surface 431 and the distal internal taper may prevent passage between surface 431 and the distal internal taper, of filtered fluid exiting passageway(s) 433 to distal outlet 404 during the urging of fluid 465 toward distal outlet 404.

Surface 431 may distally terminate as filter base end projection 437. End projection 437 may include the distal-most aspect of filter base 430.

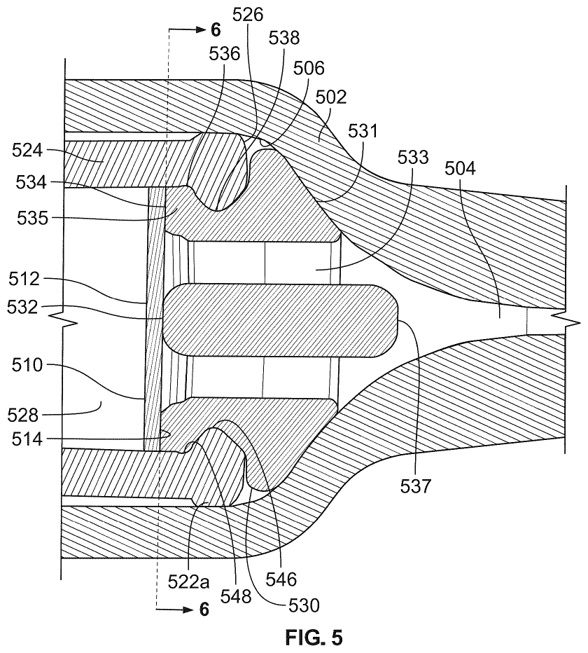

FIG. 5 will afford a partial cross-sectional view of apparatus of FIG. 4, the view taken in the plane of the page of FIG. 4 and distal to plug face 445.

FIG. 5 shows details of apparatus features that may have the same relationships, properties and functions as features of one or both of delivery devices 100 and 200 (shown in FIGS. 1 and 2, respectively) and/or be shown in FIG. 3 and/or FIG. 4, above-described, as indicated by like reference characters. These apparatus features may include container 502, distal outlet 504, inner wall 506; filter 510, central area 512, periphery 514; embedment element 522a, cylindrical body 524, distal rim 526, interior 528; filter base 530, (frusto) conical surface 531, central projection 532, passageway(s) 533, rim surface 534, rim 535, filter base convexity 536, end projection 537, filter base concavity 538; and filter cap convexity 546 and filter cap concavity 548.

FIG. 5 depicts central area 512 of filter 510 disposed closely proximal to central projection 532. Central projection 532 may support central area 512. Support of central area 512 may oppose distally directed forces acting upon filter 510 during distally directed urging of fluid within interior 528 upon and after initiation of urging of the device's fluid contents (not shown) toward distal outlet 504.

Periphery 514 of filter 510 is shown disposed closely proximal to rim surface 534. Filter 510 may be sealed to rim surface 534 along periphery 514, providing a fluid-tight seal against passage of fluid between rim surface 534 and periphery 514. Interior 528 is depicted as devoid of fluid 565; during distally directed urging of the device's fluid contents, interior 528 will contain distally directed fluid. The fluid-tight seal between rim surface 534 and periphery 514—in conjunction with fluid-tight seals between convexity 546 and concavity 538 and between convexity 536 and concavity 548; and fluid-tight seals between inner wall 506 and both (frusto) conical surface 531 and embedment element 522a—constrain distally directed fluid reaching filter 510 to flow (and be filtered) through filter 510, through passageway(s) 533, past end projection 537 and onto distal outlet 504 for delivery (and any preceding pre-delivery syringe preparation steps).

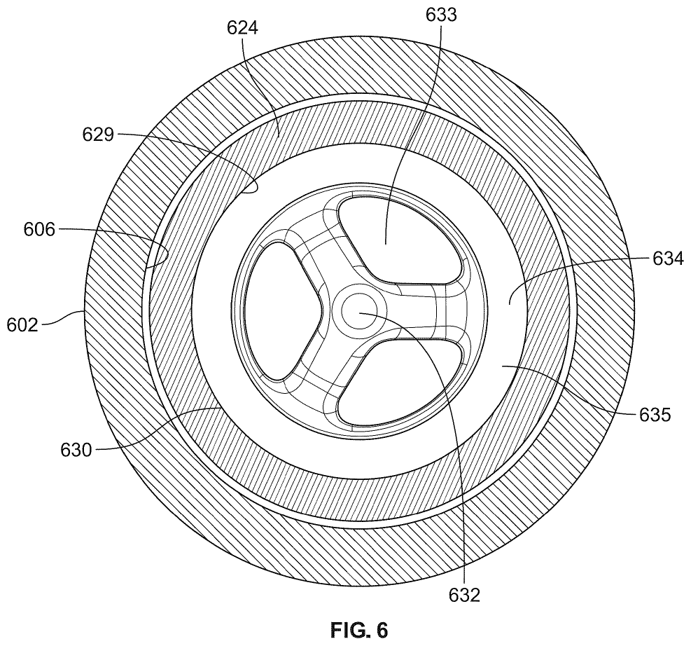

FIG. 6 will afford a view of apparatus of FIG. 5 taken distally along line 6-6, the line shown parallel and distal to filter 510.

FIG. 6 shows details of apparatus features that may have the same relationships, properties and functions as features of one or both of delivery devices 100 and 200 (shown in FIGS. 1 and 2, respectively) and/or be shown in FIG. 3, FIG. 4 and/or FIG. 5, above-described, as indicated by like reference characters. These apparatus features may include container 602, inner wall 606; cylindrical body 624, internal wall 629; and filter base 630, central projection 632, passageway(s) 633, rim surface 634 and rim 635.

FIG. 6 depicts an external circumferential aspect of rim 635 disposed closely along internal wall 629. Rim 635 may be configured to compress against internal wall 629, providing a fluid-tight seal against passage of fluid between filter base 630 and internal wall 629. The fluid-tight seal between filter base 630 and internal wall 629 may contribute to constraining the path of distal displacement of distally urged fluid to passageway(s) 633 (as discussed above in description of FIG. 5).

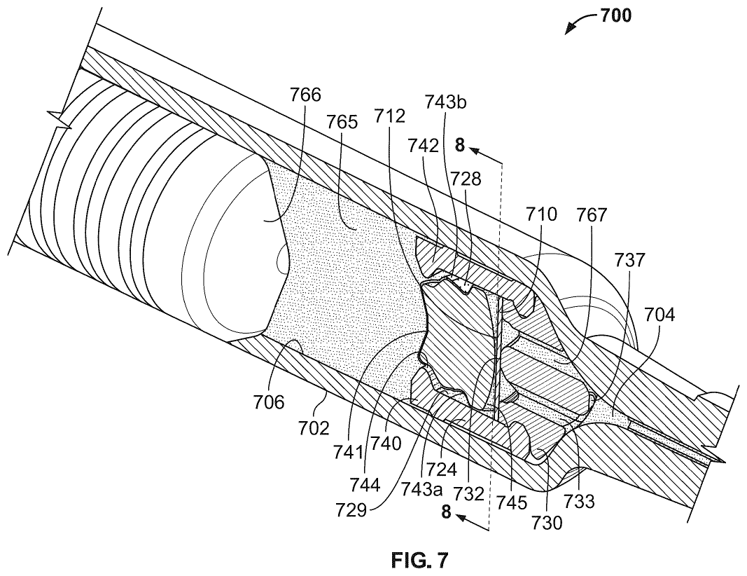

FIG. 7 shows illustrative medicament delivery device 700. Delivery device 700 may have one or more apparatus features with the same relationships, properties and functions as features of one or both of delivery devices 100 and 200 (shown in FIGS. 1 and 2, respectively) and/or be shown in FIG. 3, FIG. 4, FIG. 5 and/or FIG. 6, above-described, as indicated by like reference characters. These apparatus features may include container 702, distal outlet 704, inner wall 706; filter 710, central area 712; cylindrical body 724, interior 728, internal wall 729; filter base 730, central projection 732, passageway(s) 733, end projection 737; filter cap 740, plug 741, collar 742, groove 743a, groove rise 743b, inwardly protruding rim 744, distal face 745; and fluid 765 and plunger 766.

FIG. 7 depicts device 700 in an operational configuration that follows displacement of plug 741 from collar 742. This configuration may be deemed a filtering configuration. To achieve the depicted configuration, the user had already begun urging plunger 766 distally within container 702 toward outlet 704, effecting an increase of pressure within fluid 765. The increase of fluid pressure had exerted distally directed force upon a proximal exterior of plug 741, the plug still then being maintained within collar 742 (as shown in FIG. 4) by contact/frictional forces of interference of inwardly protruding rim 744 and groove 743a (with its groove rise 743b). (Refer to FIG. 4 for apparatus details of the interference.) Further increase of fluid pressure applied by the user via plunger 766 had then resulted in a distally directed force of sufficient magnitude to overcome the contact/frictional forces of interference, thus distally displacing plug 741 from collar 742.

To achieve the filtering configuration depicted in FIG. 7, following distal displacement of plug 741 from collar 742, plug 741 had transited interior 728 toward filter 710. Plug 741 was driven toward filter 710 by distally directed fluid 765 urged by the user's distal displacement of plunger 766 relative to inner wall 706. Distal face 745 of plug 741 was displaced distally to be disposed closely proximal to central area 712 of filter 710. With continuing distal displacement of plunger 766, fluid continued to flow through unplugged collar 742 into interior 728; between internal wall 729 and a longitudinal exterior of plug 741; along and distally away from face 745; and from there to be forced through filter 710.

FIG. 7 shows filtered fluid 767 (depicted illustratively as speckles) having emerged from, and being disposed distal to, filter 710. Filtered fluid 767 may be a filtrate of fluid 765. Filtered fluid 767 may differ in content from fluid 765. Fluid 765 may contain particulate matter of sizes that filter 710 may retain while fluid 765 is distally urged onto and into filter 710. Fluid 767 may be free of fluid 765's particulate matter retained by filter 710. The sizes of particulate matter in fluid 765 retainable by filter 710 may depend upon a pore size of filter 710. The pore size of filter 710 may be an average effective pore size. (Use in the invention of a specialty filter, as discussed above, may expand a range of distinction between filtrate and pre-filtered PFS fluid contents beyond particle size of dissolved/suspended particles, broadening the range to charge profiles and/or to molecular identities.)

As shown, filtered fluid 767 may fill passageway(s) 733; extend past end projection 737 to fill distal outlet 704; and from there to be directed for delivery of filtered fluid 767 (and for any preceding pre-delivery syringe preparation steps). As more fluid 765 is urged onto and into filter 710 by the user's further distal displacement of plunger 766 from the depicted position of plunger 766, freshly filtered fluid 767 may continue to flow distally out from filter 710, through passageway(s) 733 to distal outlet 704, and then on toward delivery.

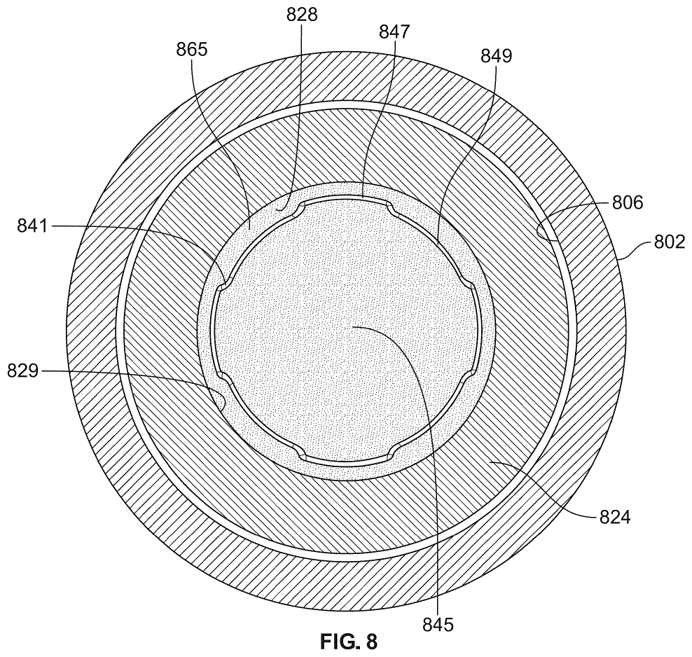

FIG. 8 will afford a view of apparatus of FIG. 7 taken proximally along line 8-8, the line shown parallel and proximal to filter 710.

FIG. 8 shows details of apparatus features that may have the same relationships, properties and functions as features of one or more of delivery devices 100, 200 and 700 (shown in FIGS. 1, 2 and 7, respectively) and/or be shown in FIG. 3, FIG. 4, FIG. 5 and/or FIG. 6, above-described, as indicated by like reference characters. These apparatus features may include container 802, inner wall 806; cylindrical body 824, interior 828, internal wall 829; plug 841, distal face 845, tab(s) 847, inter-tab gap(s) 849; and fluid 865.

FIG. 8 depicts plug 841 at least approximately centered within internal wall v 829 of cylindrical body 824. Centering of plug 841 within internal wall 829 may result from approximately equal magnitudes of distally urged fluid 865 passing along longitudinal sides of plug 841. Regions of interior 828 disposed between internal wall 829 and inter-tab gap(s) 849 may provide for greater passage of fluid 865 than may regions disposed between internal wall 829 and tab(s) 847. Geometries of plug 841, tab(s) 847, inter-tab gaps 849 and internal wall 829 may be pre-selected to achieve desired flow rates of fluid 865.

FIG. 8 shows fluid 865 disposed across distal face 845. Passage of fluid 865 along and distally away (out of the page of FIG. 8, toward a viewer) from distal face 845 may intermittently shift plug 841 relative to the filter (not shown). Intermittent shifting of plug 841 relative to the filter may contribute to dynamically changing a thickness of fluid 865 between the filter and distal face 845. Dynamically changing the thickness of fluid 865 between the filter and distal face 845 may reduce buildup of particles on a proximal side of the filter. Reducing buildup of particles on the proximal side of the filter may contribute to maintaining filtering efficiency of the filter. The thickness of fluid 865 between the filter and distal face 845 may serve to cushion the filter from direct contact with distal face 845.

Passage of fluid 865 along and distally away from distal face 845 may intermittently shift plug 841 relative to internal wall 829. Intermittent shifting of plug 841 relative to wall 829 may bring tab(s) 847 into intermittent interference with wall 829. Intermittent interference of tab(s) 847 with wall 829 may help maintain the cylinder axis (not shown) of plug 841 wobbling about approximate collinearity with longitudinal axis L. Maintenance of plug 841's cylinder axis' collinearity with axis L may preclude plug 841 becoming skewed against, and/or possibly wedged between sides of, internal wall 829.

Preclusion of plug 841 from being skewed against, and/or possibly wedged between sides of internal wall 829, was a consideration also during distal transit of plug 841 within interior 828 following distal displacement of plug 841 from within the collar (not shown) of cylindrical body 824, as discussed in description of FIG. 4. A functional role of tab(s) 847 may have begun immediately upon release of plug 841 from the collar, at the outset of plug 841's distal transit into interior 828, preceding substantial flow of fluid 865 along the longitudinal exterior of plug 841. Interference of tab(s) 847 against internal wall 829 may have served to brake distally impelled plug 841 in its first moments of distally impelled travel into interior 828.

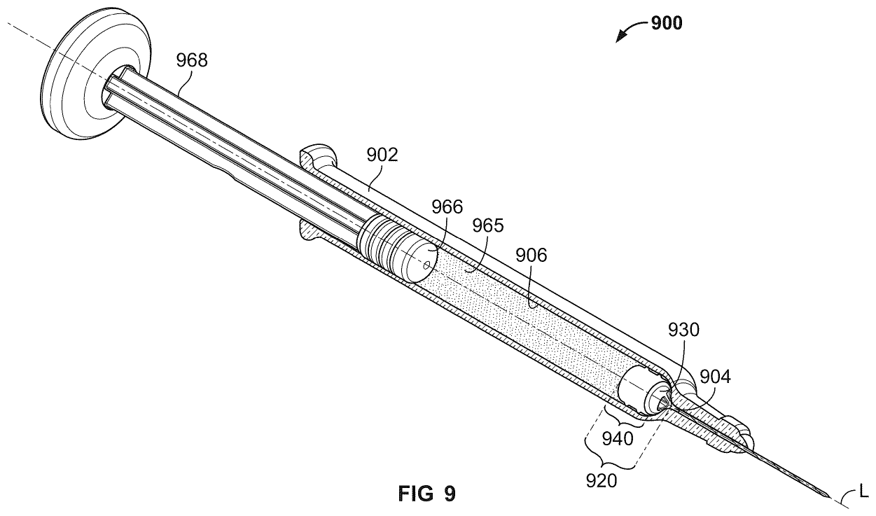

FIG. 9 shows illustrative medicament delivery device 900. Delivery device 900 may have one or more apparatus features with the same relationships, properties and functions as features of one or more of delivery devices 100, 200 and 700 (shown in FIGS. 1, 2 and 7, respectively) and/or be shown in FIG. 3, FIG. 4, FIG. 5, FIG. 6 and/or FIG. 8, above-described, as indicated by like reference characters. These apparatus features may include container 902, distal outlet 904, inner wall 906; filter housing 920; filter base 930; and fluid 965, plunger 966 and plunger rod 968.

Device 900 is shown in a pre-filtering operational configuration, with depiction of distal hollow sections of filter base 930 and of distal outlet 904 as devoid of fluid.

Filter housing 920 may include filter base 930 and filter cap 940. Filter cap 940 may, similarly to previously illustrated and discussed filter caps, be configured to block fluid access to a filter (not shown) in the pre-filtering operational configuration of device 900; and to allow fluid access to the filter in a filtering operational configuration of device 900. Filter cap 940 may be different in structure and mode of operation from filter caps previously illustrated in and/or discussed in reference to FIGS. 2-8.

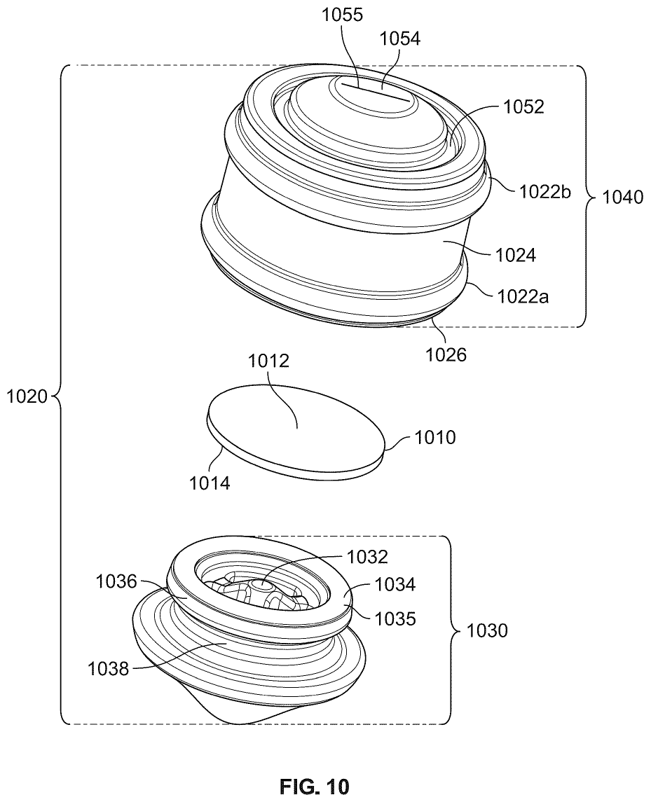

Filter caps previously illustrated in and/or discussed in reference to FIGS. 2-8 may be deemed displaceable plug filter caps. Filter cap 940 may not be a displaceable plug filter cap. Filter cap 940 may be deemed an openable diaphragm filter cap. The openable diaphragm filter cap, as a category, may include a burstable diaphragm filter cap. Differences in structure and mode of operation of openable diaphragm filter caps from displaceable plug filter caps will be illustrated in and/or discussed in reference to FIG. 10-14.

FIG. 10 shows details of apparatus features that may have the same relationships, properties and functions as features of one or more of delivery devices 100, 200, 700 and 900 (shown in FIGS. 1, 2, 7 and 9, respectively) and/or be shown in FIG. 3, FIG. 4, FIG. 5, FIG. 6 and/or FIG. 8, above-described, as indicated by like reference characters. These apparatus features may include filter 1010, central area 1012, periphery 1014; filter housing 1020, embedment elements 1022a and 1022b (collectively, embedment elements 1022), hollow cylindrical body 1024, distal rim 1026; filter base 1030, proximal central projection 1032, proximal rim surface 1034, proximal rim 1035, circumferential convexity 1036 and circumferential concavity 1038; and filter cap 1040.

The exploded view of FIG. 10 depicts filter base 1030 and filter cap 1040 separate from each other, with filter 1010 spaced in-between. FIG. 10 may afford unobstructed views of features of filter housing 1020 that may be more difficult to discern in views of assembled filter housing 1020 (such as filter housing 920, shown in FIG. 9).

Convexity 1036 and/or concavity 1038 may be configured to mechanically interfere with distal interior features (not shown) of filter cap 1040. The distal interior features of cap 1040 may have the same relationships, properties and functions as distal interior features of one or more of delivery devices 200 and 700 (shown in FIGS. 1 and 2, respectively) and/or be shown in FIG. 4, FIG. 5, FIG. 6, FIG. 7 and/or FIG. 8, above-described.

Interference of convexity 1036 and/or concavity 1038 with the distal interior features of filter cap 1040 may fit together filter base 1030 and filter cap 1040. Interference of convexity 1036 and/or concavity 1038 with the distal interior features of filter cap 1040 may secure together filter base 1030 and filter cap 1040.

Convexity 1036 and/or concavity 1038 may be configured to compress against the distal interior features of filter cap 1040. Compression of convexity 1036 and/or concavity 1038 against the distal interior features of filter cap 1040 may provide a fluid-tight seal between filter base 1030 and filter cap 1040. The distal interior features of filter cap 1040 may be configured to compress against convexity 1036 and/or concavity 1038. Compression of the distal interior features of filter cap 1040 against convexity 1036 and/or concavity 1038 may provide a fluid-tight seal between filter base 1030 and filter cap 1040.

Distal rim 1026 may be configured to receive at least portions of filter base 1030. Distal rim 1026 may be configured to longitudinally receive filter 1010 and proximal rim 1035.

As shown, embedment element 1022b may lie along a circumferential exterior of cylindrical body 1024. Embedment element 1022b may extend along the full circumference of the circumferential exterior of cylindrical body 1024. Embedment element 1022b may not extend along a full circumference of the circumferential exterior of cylindrical body 1024.

Embedment elements 1022 may compress against an inner container wall of the device (not shown). Compression of embedment elements 1022 against the inner container wall may provide fluid-tight sealing against passage of fluid between embedment elements 1022 and the inner container wall.

Filter cap 1040 may define a cylinder axis (not shown). The cylinder axis may be centered with respect to the circumference of the circumferential exterior of cylindrical body 1024. The cylinder axis may be disposed parallel to a longitudinal exterior of cylindrical body 1024. The cylinder axis may be disposed parallel to, and centered with respect to, a cylindrical internal wall (not shown) of cylindrical body 1024. The cylinder axis may be centered with respect to a circumference of distal rim 1026. The cylinder axis may intersect a center of the circumference of diaphragm 1054. The cylinder axis may be disposed at least approximately coaxially with the device longitudinal axis (not shown).

Structural differences of filter cap 1040 from displaceable plug filter caps may be disposed in a proximal portion of filter cap 1040. The proximal portion of filter cap 1040 may include diaphragm ring support 1052. Ring support 1052 may be disposed along a proximal exterior rim of filter cap 1040. Ring support 1052 may be disposed along all the proximal exterior rim. Ring support 1052 may be disposed within the proximal exterior rim. Ring support 1052 may be disposed in an inner circumference of the proximal exterior rim. Ring support 1052 may span all the inner circumference of the proximal exterior rim. Ring support 1052 may be integral to the proximal exterior rim. The proximal exterior rim may support ring support 1052.

Ring support 1052 may support diaphragm 1054. Diaphragm 1054 may be integral to an inner circumference of ring support 1052. Diaphragm 1054 may span all the inner circumference of ring support 1052. Diaphragm 1054 may fully occupy a region concentric with the inner circumference of ring support 1052.

Diaphragm 1054 may include diaphragm slit 1055. The cylinder axis of cylindrical body 1024 may intersect slit 1055. The cylinder axis may run through a midpoint of a length of slit 1055. Slit 1055 may include a plurality of slits.

Slit 1055 may traverse an area of diaphragm 1054 along a secant of diaphragm 1054. Slit 1055 may traverse an area of diaphragm 1054 along a diameter of diaphragm 1054. Slit 1055 may be disposed along the diameter of diaphragm 1054. Slit 1055 may be disposed along all the diameter of diaphragm 1054. Slit 1055 may be disposed along a portion of the diameter of diaphragm 1054.

Slit 1055 may traverse all the depth of a full thickness of diaphragm 1054. Slit 1055 may traverse part of the depth of the full thickness of diaphragm 1054. Slit 1055 may extend into diaphragm 1054 from a proximal face of diaphragm 1054, distally traversing part of the depth of the full thickness of diaphragm 1054. Slit 1055 may extend into diaphragm 1054 from a distal face (not shown) of diaphragm 1054, proximally traversing part of the depth of the full thickness of diaphragm 1054.

Diaphragm 1054 may be configured, in a pre-filtering configuration, to prevent passage of PFS fluid to filter 1010. Diaphragm 1054 may be configured, in a transition from the pre-filtering configuration to a filtering configuration, to be unsealed by a distally directed urging of the PFS fluid against a proximal face of diaphragm 1054. Diaphragm 1054 may be configured, in a filtering configuration, to provide passage to a flow of the PFS fluid contents through an unsealed diaphragm 1054 to filter 1010.

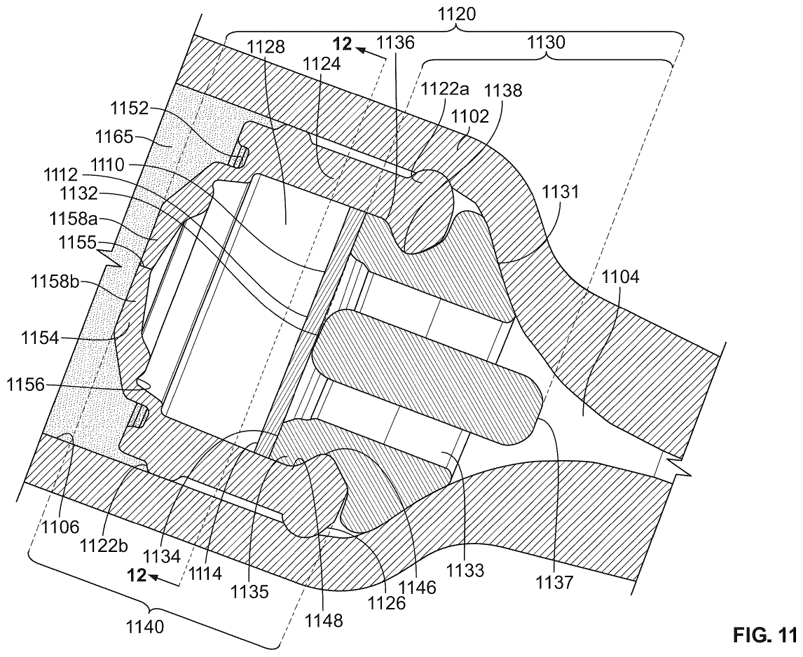

FIG. 11 will afford a partial cross-sectional view of a distal portion of apparatus of FIG. 9.

FIG. 11 shows details of apparatus features that may have the same relationships, properties and functions as features of one or more of delivery devices 100, 200, 700 and 900 (shown in FIGS. 1, 2, 7 and 9, respectively) and/or be shown in FIG. 3, FIG. 4, FIG. 5, FIG. 6, FIG. 8 and/or FIG. 10, above-described, as indicated by like reference characters. These apparatus features may include container 1102, distal outlet 1104, inner wall 1106; filter 1110, central area 1112, periphery 1114; filter housing 1120, embedment elements 1122 (embedment element 1122a and embedment element 1122b) , cylindrical body 1124, distal rim 1126, interior 1128; filter base 1130, (frusto) conical surface 1131, central projection 1132, passageway(s) 1133, rim surface 1134, rim 1135, filter base convexity 1136, end projection 1137, filter base concavity 1138; filter cap 1140, filter cap convexity 1146, filter cap concavity 1148; diaphragm ring support 1152, diaphragm 1154, diaphragm slit 1155; and fluid 1165.

In FIG. 11's depicted pre-filtering operational configuration, interior 1128 of cylindrical body 1124, passageway(s) 1133 and distal outlet 1104, are shown devoid of fluid 1165. Slit 1155 may be closed against a flow of fluid 1165 through diaphragm 1154, preventing the distal flow of fluid 1165 into interior 1128 toward filter 1110 and distally beyond.

Diaphragm 1154 may include diaphragm flaps. The diaphragm flaps may include diaphragm flap 1158a. The diaphragm flaps may include diaphragm flap 1158b. The diaphragm flaps may be closed against each other at slit 1155. The diaphragm flaps may be sealed against each other at slit 1155.

The diaphragm flaps being closed and/or sealed against each may provide a fluid-tight sealing against distal displacement of fluid 1165 into interior 1128. An increase of fluid pressure applied by the user in initiation of transitioning to a filtering operational configuration of the delivery device, may achieve a pressure within fluid 1165 sufficient to overcome forces closing and/or sealing slit 1155 against distal fluid displacement through diaphragm 1154. Diaphragm 1154 may be opened at slit 1155 by the user-applied pressure. Diaphragm 1154 may be burst open at slit 1155 by the user-applied pressure.

In the filtering configuration (not shown) achieved with slit 1155 opened/burst by increased user-applied pressure within fluid 1165, distally urged fluid may flow into interior 1158. The distally urged fluid may drive flap 1158a and/or flap 1158b to fold into cylinder body 1124 Folded flap 1158a and/or flap 1158b may extend into interior 1128. Folded flap 1158a and/or flap 1158b may extend into interior 1128 at an acute angle(s) from the depicted positions of the flap(s). Folded flap 1158a and/or flap 1158b may extend into interior 1128 at least approximately perpendicularly from the depicted positions of the flap(s). Folded flap 1158a and/or flap 1158b may extend into interior 1128 at an obtuse angle(s) from the depicted positions of the flap(s).

Flap 1158a and/or flap 1158b may fold at hinge elements 1156. Hinge elements 1156 may be supported by ring support 1152. Hinge elements 1156 may include regions along encompassed by a circumference of a distal face of diaphragm 1154. The regions encompassed by the circumference of the distal face of diaphragm 1154 may include grooves extending proximally into a material of diaphragm 1154. Corresponding regions lying along a proximal face of diaphragm 1154 may include grooves extending distally (not shown) into the material of diaphragm 1154. The grooves extending into the material of the diaphragm may be disposed along line segments encompassed by support ring 1152. The grooves extending into the material of the diaphragm may be disposed along arcs concentric with cylindrical body 1124. The material of diaphragm 1154 disposed along the grooves may be more likely to fold than the material of diaphragm 1154 disposed away from the grooves.

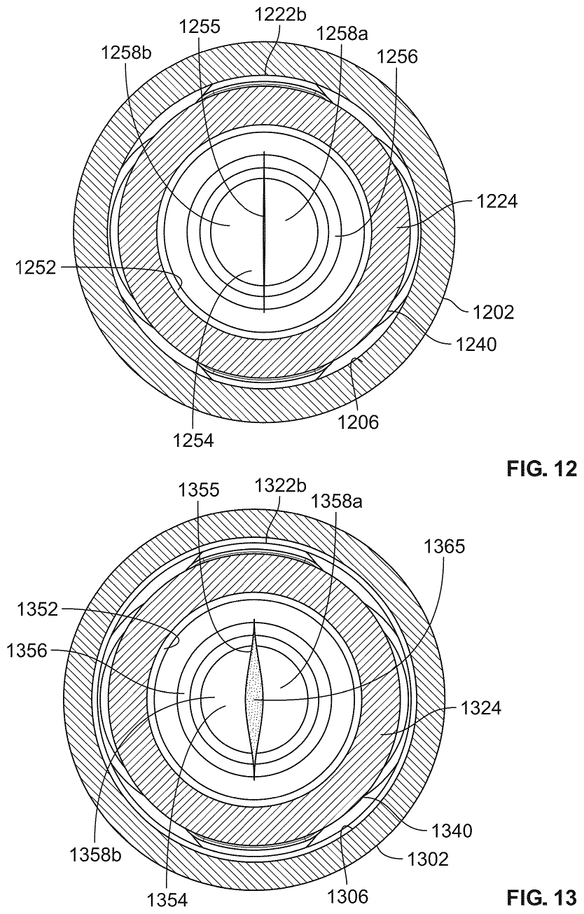

FIG. 12 will afford a view of apparatus of FIG. 11 taken proximally along line 12-12, the line shown running through interior 1128, the line disposed parallel and proximal to filter 1110.

FIG. 12 shows details of apparatus features that may have the same relationships, properties and functions as features of one or more of delivery devices 100, 200, 700 and 900 (shown in FIGS. 1, 2, 7 and 9, respectively) and/or be shown in FIG. 3, FIG. 4, FIG. 5, FIG. 6, FIG. 8, FIG. 10 and/or FIG. 11, above-described, as indicated by like reference characters. These apparatus features may include container 1202, inner wall 1206; embedment element 1222b, cylindrical body 1224; filter cap 1240; and diaphragm ring support 1252, diaphragm 1254, diaphragm slit 1255, diaphragm hinge elements 1256, and diaphragm flaps 1258a and 1258b.

Embedment element 1222b may compress against inner wall 1206. Embedment element 1222b compressing against inner wall 1206 may contribute to lodging of filter cap 1240 within container 1202. Embedment element 1222b compressing against inner wall 1206 may contribute to filter cap 1240 being lodged with the cylindrical axis of cylindrical body at least approximately coaxial with the device axis (neither axis shown).

Embedment Element 1222b Compressing Against Inner wall 1206 may contribute to sealing of embedment element 1222b against inner wall 1206. Sealing of embedment element 1222b against inner wall 1206 may provide a fluid-tight seal against passage of PFS fluid contents (not shown) distally (i.e., out of the page of FIG. 12, toward the viewer) between embedment element 1222b and inner wall 1206.

In FIG. 12's depicted pre-filtering operational configuration, a distal face of diaphragm 1254 is shown. The distal face of diaphragm 1254 may be a face of diaphragm 1254 interior to cylindrical body 1224 (as shown in FIG. 11).

Facing edges of flaps 1258a and 1258b may be closed and/or sealed against each other along slit 1255, providing diaphragm 1254 a fluid-tight seal against distal passage through diaphragm 1254 of PFS fluid contents into cylindrical body 1224.

FIG. 13 will afford a view of apparatus of FIG. 12 with the same view as taken in FIG. 12, but at the outset of a filtering operational configuration in which distally urged PFS fluid contents have just opened/burst the diaphragm.

FIG. 13 shows details of apparatus features that may have the same relationships, properties and functions as features of one or more of delivery devices 100, 200, 700 and 900 (shown in FIGS. 1, 2, 7 and 9, respectively) and/or be shown in FIG. 3, FIG. 4, FIG. 5, FIG. 6, FIG. 8, FIG. 10, FIG. 11 and/or FIG. 12, above-described, as indicated by like reference characters. These apparatus features may include container 1302, inner wall 1306; and embedment element 1322b cylindrical body 1324; filter cap 1340; diaphragm ring support 1352, diaphragm 1354, diaphragm slit 1355, diaphragm hinge elements 1356, and diaphragm flaps 1358a and 1358b; and fluid 1365.

FIG. 13 depicts an early stage of a filtering operational configuration at which fluid 1365, urged distally within container 1302 against an exterior surface (not shown) of diaphragm 1354, may have attained sufficient pressure to open/burst diaphragm slit 1355. Upon diaphragm slit 1355 having opened/burst in response to the pressure, fluid 1365 is depicted starting to enter a space between facing edges of flap 1358a and flap 1358b. Flaps 1358a and 1358b may be bending away from each other. Flaps 1358a and 1358b may be starting to fold distally into cylinder 1324. Flaps 1358a and 1358b may be starting to fold along hinge elements 1356. Hinge elements 1356 may be supported by ring support 1352.

Diaphragm 1354 may be configured to reseal if a force of the distal urging of fluid 1365 is diminished below that required to generate the pressure sufficient to open/burst slit 1355, with facing edges of flap 1358a and flap 1358b then approaching and re-closing against each other, sealing the slit. Diaphragm 1354 may be configured to remain open/burst even if, after the slit is open/burst, the force of the distal urging of fluid 1365 is diminished below that which had been required to generate the pressure sufficient to open/burst slit 1355. Diaphragm 1354 may be configured such that before a particular pre-selected stage of opening of slit 1355 and distal folding of flaps 1358a and 1358b into cylinder 1324, diaphragm 1354 will re-close upon diminishment of fluid pressure. Diaphragm 1354 may be configured such that after a particular pre-selected stage of opening of slit 1355 and distal folding of flaps 1358a and 1358b into cylinder 1324, diaphragm 1354 will not re-close even upon diminishment of the fluid pressure.

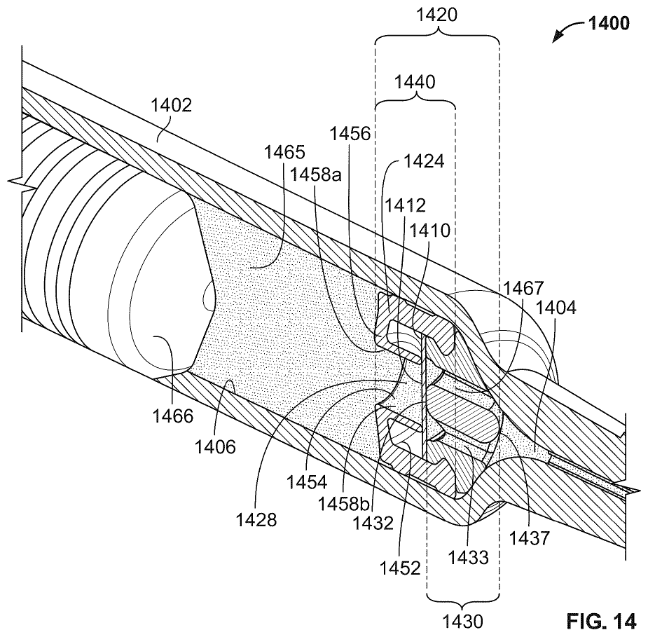

FIG. 14 shows illustrative medicament delivery device 1400. Delivery device 1400 may have one or more apparatus features with the same relationships, properties and functions as features of one or more of delivery devices 100, 200, 700 and 900 (shown in FIGS. 1, 2, 7 and 9, respectively) and/or be shown in FIG. 3, FIG. 4, FIG. 5, FIG. 6, FIG. 8, FIG. 10, FIG. 11, FIG. 12 and/or FIG. 13, above-described, as indicated by like reference characters. These apparatus features may include container 1402, distal outlet 1404, inner wall 1406; filter 1410, central area 1412; filter housing 1420, cylindrical body 1424, interior 1428; filter base 1430, central projection 1432, passageway(s) 1433, end projection 1437; filter cap 1440; diaphragm ring support 1452, diaphragm 1454, diaphragm hinge elements 1456, diaphragm flap 1458a, diaphragm flap 1458b; and fluid 1465, plunger 1466 and fluid 1467.

FIG. 14 depicts device 1400 in an operational filtering configuration that follows opening/bursting of the diaphragm slit (shown in FIG. 13) by the user having already urged plunger 1466 distally within container 1402 toward outlet 1404, producing a pressure within fluid 1465 sufficient to open/burst diaphragm 1454. (For the shift from pre-filtering configuration to the start of the filtering configuration, compare FIGS. 12 and 13.) To achieve the filtering configuration depicted in FIG. 14, following opening/bursting of diaphragm 1454 the user had continued distal displacement of plunger 1466, driving fluid 1465 to flow through opened/burst diaphragm 1454 into interior 1428; between distally folded diaphragm flaps 1458a and 1458b; o a proximal face of filter 1410. Proximal projection 1432 provided support to central area 1412, supporting filter 1410 against forces suddenly applied to filter 1410 by distally impelled fluid 1465 first encountering the proximal face of filter 1410. With distally impelled fluid 1465 continuing to encounter the proximal face of filter 1410, fluid 1465 was forced through and filtered by filter 1410.

FIG. 14 shows filtered fluid 1467 having emerged from, and being disposed distal to, filter 1410. Filtered fluid 1467 may be a filtrate of fluid 1465. Filtered fluid 1467 may differ in content from fluid 1465. Fluid 1465 may contain particulate matter of sizes that filter 1410 may retain while fluid 1465 is distally urged onto and into filter 1410. Fluid 1467 may be free of fluid 1465's particulate matter retained by filter 1410. The sizes of particulate matter in fluid 1465 retainable by filter 1410 may depend upon a pore size of filter 1410. The pore size of filter 1410 may be an average effective pore size. (Use in the invention of a specialty filter, as discussed above, may expand a range of distinction between filtrate and pre-filtered PFS fluid contents beyond particle size of dissolved/suspended particles, broadening the range to charge profiles and/or to molecular identities.) Diaphragm flaps 1458a and 1458b are depicted having been folded into interior 1428, with their distal ends (which prior to opening/bursting of diaphragm 1410, had been flap edges that faced each other across, and had closed/sealed, the diaphragm's slit) facing and proximal to the proximal surface of filter 1410. The folding of diaphragm flaps 1458a and 1458b had occurred at diaphragm hinge elements 1456, with diaphragm hinge elements 1456 supported by diaphragm ring support 1452. Filtering of fluid 1465 to produce fluid 1467 may be effected also with less extreme folding than depicted of diaphragm flaps 1458a and 1458b into interior 1428.

As shown, filtered fluid 1467 may fill passageway(s) 1433; extend past end projection 1437 to fill distal outlet 1404; and from there to be directed for delivery of filtered fluid 1467 (and for any preceding pre-delivery syringe preparation steps). As more fluid 1465 is urged onto and into filter 1410 by the user's further distal displacement of plunger 1466 from the depicted position of plunger 1466, freshly filtered fluid 1467 may continue to flow distally out from filter 1410, through passageway(s) 1433 to distal outlet 1404, and then on toward delivery.

After cessation of flow of distally impelled fluid 1465 through interior 1428 to filter 1410, diaphragm flaps 1458a and 1458b may unfold back toward the positions they occupied in the pre-filtering configuration (such as shown in FIGS. 11 and 12). The extent of such unfolding may be set by preselection of materials, structures and geometries of diaphragm 1454, diaphragm flap 1458a and 1458b, diaphragm ring support 1452 and diaphragm hinge elements 1456. Such unfolding may re-close diaphragm 1454.

Thus, apparatus and methods for filtering PFS fluid contents close in time before medicament delivery in a fashion that maintains the advantages of PFS use while sequestering the filter from the fluid until initiation of the delivery, have been provided, with methods including manufacturing methods. Persons skilled in the art will appreciate that the present invention can be practiced by other than the described embodiments, which are presented for purposes of illustration rather than of limitation. The present invention is limited only by the claims that follow.

1. A fluid delivery device for delivery of a filtered fluid from a distal outlet of the device, the device defining a longitudinal axis and comprising:
fluid within a fluid container, the container disposed coaxial with the longitudinal axis; and
a filter embedded within the container, the filter disposed:
between the distal outlet and the fluid; and
athwart the longitudinal axis.

2. The delivery device of claim 1 further comprising a filter housing that includes a proximal filter cap and a distal filter base.

3. The delivery device of claim 2 wherein:
the filter is fixed in the filter housing; and
the filter housing is lodged in the container.

4. The delivery device of claim 2 wherein the filter base is configured to provide distal support to the filter.

5. The delivery device of claim 4 wherein the distal support is provided to a central area of the filter by a proximal central projection of the filter base.

6. The delivery device of claim 5 wherein the filter is not affixed to the proximal central projection.

7. The delivery device of claim 2 wherein a periphery of the filter is affixed to a proximal surface of the filter base.

8. The delivery device of claim 2 wherein a region of the filter circumferentially bordering a filter perimeter is joined, in a fluid-tight seal, to a proximal surface of the filter base, the seal configured to prevent passage of the fluid between an interior of the filter base and any of the filter perimeter.

9. The delivery device of claim 2 wherein the filter base is secured to the filter cap.

10. The delivery device of claim 2 wherein interference between at least one filter cap convexity and at least one filter base concavity is configured to secure the filter cap to the filter base.

11. The delivery device of claim 10 wherein the at least one filter base concavity and the at least one filter cap convexity are contoured at least approximately complementarily to each other.

12. The delivery device of claim 10 wherein:
the at least one filter cap convexity is disposed along a portion of an interior surface of the filter cap; and the at least one filter base concavity is disposed along a portion of an exterior surface of the filter base.

13. The delivery device of claim 2 wherein interference between at least one filter cap surface convexity and at least one filter base surface concavity is configured to provide a fluid-tight seal.

14. The delivery device of claim 2 wherein interference between at least one filter cap surface concavity and at least one filter base surface convexity is configured to provide a fluid-tight seal.

15. The device of claim 2 wherein a wall of the filter housing is configured to seal against an inner wall of the container, thereby preventing passage of the fluid between an exterior of the filter housing and the inner wall.

16. The delivery device of claim 15 wherein the wall includes a filter cap sealing flange disposed along an external periphery of the filter cap.

17. The delivery device of claim 16 wherein the filter cap sealing flange includes a plurality of filter cap sealing flanges.

18. The delivery device of claim 15 wherein the wall includes a filter base sealing surface supported by a distal section of a body of the filter base.

19. The delivery device of claim 1 wherein an average pore size of the filter is about 1 micron to about 10 microns.

20. The delivery device of claim 19 wherein the average pore size is about 4 microns to about 6 microns.

21. The delivery device of claim 1 wherein an average pore size of the filter is about 0.01 micron to about 1 micron.

22. The delivery device of claim 21 wherein the average pore size is about 0.1 microns to about 0.3 micron.

23. The device of claim 1 further comprising a filter housing configured to:
prevent the fluid from contacting the filter prior to an urging of the fluid toward the distal outlet; and
allow the fluid to contact the filter following initiation of the urging.

24. The delivery device of claim 1 further comprising:
a slidable plunger proximal the fluid, the plunger configured to:
seal the fluid within the container; and
urge the fluid toward the distal outlet; and,
a filter cap configured, prior to the delivery, to block fluid access to the filter;
wherein the fluid is configured, upon urging by the plunger, to unblock the cap to a flow of the fluid-toward the distal outlet.

25. The delivery device of claim 2 wherein the filter cap includes a proximal plug disposed in and sealing a collar against flow of the fluid to the filter, the plug configured to:
be distally displaced, relative to the collar, by distally urged fluid; and, thereby,
unseal the collar to the flow of the fluid therethrough.

26. The delivery device of claim 25 wherein the plug includes, along an exterior of the plug, a plug groove extending radially inward.

27. The delivery device of claim 26 wherein the collar includes, along an interior surface of the collar, a collar rim extending radially inward.

28. The delivery device of claim 27 wherein, prior to the delivery, the plug seals the collar against the flow by interference between the plug groove and the collar rim.

29. The delivery device of claim 28 wherein the plug;
defines a cylinder axis that, prior to delivery, is disposed at least approximately coaxially with the longitudinal axis; and
includes, distal the plug groove, at least one tab extending radially outward orthogonal the cylinder axis, with an inter-tab gap disposed circumferentially along an exterior of the plug.

30. The delivery device of claim 29 wherein:
the at least one tab is a plurality of tabs; and
the inter-tab gap is disposed between circumferentially adjacent facing lateral edges of circumferentially adjacent tabs.

31. The delivery device of claim 29 wherein:
the at least one tab is one tab; and
the inter-tab gap is disposed between circumferentially adjacent facing lateral edges of the one tab.

32. The delivery device of claim 29 wherein the at least one tab is configured to maintain an orientation of the cylinder axis at least approximately coaxial with the longitudinal axis after distal displacement of the plug relative to the collar.

33. The delivery device of claim 29 wherein the filter cap includes a cylindrical body that includes the collar disposed at a proximal end of the body.

34. The delivery device of claim 33 wherein the inter-tab gap is configured to allow the flow of the fluid between the exterior of the plug and an interior of the cylindrical body after distal displacement of the plug relative to the collar into the interior of the cylindrical body.

35. The delivery device of claim 2 wherein the filter cap includes an initially closed diaphragm preventing flow of the fluid to the filter, the diaphragm configured to:
be opened by distally urged fluid; and, thereby,
provide passage to the flow of the fluid.

36. The delivery device of claim 35 wherein the diaphragm is a slit diaphragm.

37. The delivery device of claim 36 wherein the slit diaphragm includes a single slit.

38. The delivery device of claim 36 wherein the slit diaphragm includes a plurality of slits.

39. The delivery device of claim 35 wherein the diaphragm is configured to re-close after being opened.

40. The delivery device of claim 35 wherein the diaphragm is configured to re-close after cessation of the flow of the fluid.

41. The delivery device of claim 35 wherein the diaphragm is configured to re-close after cessation of distal urging of the fluid.

42. The delivery device of claim 1 wherein the fluid includes a medicament.

43. The delivery device of claim 1 wherein the container includes a syringe barrel.

44. The delivery device of claim 1 wherein a wall of the container includes a plastic polymer.

45. A filter housing for embedment within a medicament container of a device, the device defining a longitudinal axis, the device for delivery of a filtered medicament from a distal outlet of the device, the filter housing comprising:
a filter base configured to support a filter, the filter:
disposed upon the base; and
configured to prevent passage therethrough of particles larger than a largest pore size of the filter; and
a filter cap configured to join with the base;
wherein the filter housing is configured to be embedded in the container proximal the distal outlet.

46. The filter housing of claim 45 wherein the filter housing is further configured to provide a seal of a perimeter of the filter, the perimeter, when sealed, configured to prevent passage therearound of the medicament.

47. The filter housing of claim 45 wherein an exterior of the filter housing is configured to provide a seal against an inner wall of the container, the seal configured to prevent distal flow of the medicament between the inner wall and the filter housing.

48. The filter housing of claim 45 wherein the filter cap is further configured to:
prior to the delivery of the medicament, block access to the filter by the medicament, the medicament disposed:
within the container:
proximal the filter; and
distal a longitudinally slidable plunger that is configured to:
seal against an inner wall of the container; and
urge the medicament toward the distal outlet; and
in contact with a proximal exterior aspect of the cap; and
be unblocked by distal urging of the medicament upon the aspect, thereby producing an unblocked cap, the unblocked cap allowing a flow of the medicament to the filter.

49. The filter housing of claim 45 wherein the filter includes acrylic polymer.

50. The filter housing of claim 45 wherein the filter includes polyethersulfone.

51. The filter housing of claim 45 wherein the filter includes polyvinylidene fluoride.

52. The filter housing of claim 45 wherein an average pore size of the filter is about 5 microns.

53. The filter housing of claim 45 wherein an average pore size of the filter is about 0.2 microns.

54. The filter housing of claim 48 wherein the aspect includes a proximal plug disposed in, and sealing, a collar of the filter cap, the plug configured to:
be distally displaced, relative to the collar, by distally urged medicament; and, thereby,
unseal the collar to the flow of the medicament therethrough.

55. The filter housing of claim 48 wherein the aspect includes an initially closed diaphragm configured to:
be opened by distally urged medicament; and, thereby,
provide passage to the flow of the medicament.

56. A method of manufacturing a medicament delivery device for delivery of a filtered medicament, the device defining a longitudinal axis, the method comprising:
providing a medicament container disposed coaxially with the axis, the container having a distal outlet and a proximal
assembling a filter housing, the assembling including:
providing a filter base configured to support a filter;
disposing the filter upon a face of the base;
sealing a perimeter of the filter against fluid flow;
joining the base with a filter cap, the cap including:
a rib configured to seal against an inner wall of the container; and
an exterior aspect configured to:
block fluid access to the filter before the delivery; and
be unblocked to provide fluid access to the filter during the delivery;
introducing the filter housing into the container through the proximal opening, with the base disposed distally and the exterior aspect disposed proximally relative to the axis;
embedding the filter housing in the container proximal the distal outlet;
sealing the rib against the inner wall; and,
via the proximal opening:
transferring the medicament to the container; and,
inserting a longitudinally slidable plunger, the plunger configured to:
seal the medicament within the container; and
urge the medicament toward the distal outlet.

57. The method of claim 56 wherein the inserting is performed without distally urging the medicament sufficiently to unblock the exterior aspect.

58. The method of claim 56 wherein the housing includes an interior surface configured to seal the filter perimeter.

59. The method of claim 56 wherein the exterior aspect includes a proximal plug disposed in, and sealing, a collar of the filter cap, the plug configured to:
be distally displaced, relative to the collar, by distally urged medicament; and, thereby,
unseal the collar to flow of the medicament therethrough.

60. The method of claim 56 wherein the exterior aspect includes an initially closed slit diaphragm configured to:
be opened by distally urged medicament; and, thereby,
provide passage to flow of the medicament.
